(19) United States
(12) Patent Application Publication
(10) Pub. No.: US 2021/0039062 A1
(43) Pub. Date: Feb. 11, 2021

(54) NANOPATTERNING FOR CONTROLLING CELL CYTOSKELETON

(71) Applicant: NORTHWESTERN UNIVERSITY, Evanston, IL (US)

(72) Inventors: Chad A. Mirkin, Wilmette, IL (US); Milan Mrksich, Hinsdale, IL (US); Brian R. Meckes, Highland Village, TX (US); Maria D. Cabezas, Evanston, IL (US)

(21) Appl. No.: 16/975,987

(22) PCT Filed: Feb. 27, 2019
(86) PCT No.: PCT/US19/19825

§ 371 Date: Aug. 26, 2020

Related U.S. Application Data
(60) Provisional application No. 62/635,928, filed on Feb. 27, 2018.

(51) Int. Cl. B01J 19/00; C12N 5/0775

(52) U.S. Cl. CPC B01J 19/0046; C12N 5/0662; B01J 2219/00317; C12N 2535/10; B01J 2219/00637; B01J 2219/00659; B01J 2219/00743; B01J 2219/00617

(57) ABSTRACT

The present disclosure relates to nanolithographical cell patterning. In some aspects, the present disclosure provides materials and methods for making an oriented array.

### CROSS REFERENCE TO RELATED APPLICATIONS

This application claims the priority benefit under 35 U.S.C. § 119(e) of U.S. Provisional Patent Application No. 62/635,928, filed Feb. 27, 2018, the disclosure of which is incorporated herein by reference in its entirety.

### STATEMENT OF GOVERNMENT SUPPORT

This invention was made with government support under FA9550-17-1-0348 awarded by the Air Force Office of Scientific Research; U54CA151880 awarded by the National Institutes of Health; and U54CA199091 awarded by the National Institutes of Health. The government has certain rights in the invention.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: Filename: 2018-027 Seqlisting.txt; Size: 811 bytes, created: Feb. 27, 2019.

### BACKGROUND

The organization of the extracellular matrix (ECM) at both nano and micro length scales directs the formation of focal adhesion (FA) complexes in adherent cells and in turn influences cytoskeletal organization, cell shape, and specific cellular responses such as migration, survival, and differentiation [Delon et al., Curr. Opin. Cell Biol. 19, 43-50 (2007); Frantz et al., J. Cell Sci. 123, 4195-4200 (2010); Geiger et al., Nat. Rev. Mol. Cell Biol. 10, 21-33 (2009)]. The development of methods that can pattern protein ligands to solid substrates has been important to control cell adhesion [Lohmuller et al., Biointerphases 6, MR1-12 (2011); Arnold et al., Nano Lett. 8, 2063-2069 (2008); Cavalcanti-Adam et al., HFSP J. 2, 276-285 (2008); Huang et al., Nano Lett. 9, 1111-1116 (2009); Selhuber-Unkel et al., Biophys. J. 98, 543-551 (2010); Vignaud et al., J. Cell Sci. 125, 2134-2140 (2012); Mrksich et al., Proc. Natl. Acad. Sci. U.S.A. 93, 10775-10778 (1996); Chen et al., Biotechnol. Prog. 14, 356-363 (1998); Thery et al., Cell Motil. Cytoskelet. 63, 341-355 (2006); Jain et al., Proc. Natl. Acad. Sci. U. S. A. 110, 11349-11354 (2013)] and to understand the many ways in which cells respond to ECM organization at a range of length scales [Lohmuller et al., Biointerphases 6, MR1-12 (2011); Mrksich et al., Annu. Rev. Biophys. Biomol. Struct. 25, 55-78 (1996); Lee et al., J. Am. Chem. Soc. 125, 5588-5589 (2003); Lim et al., Angew. Chem., Int. Ed. 42, 2309-2312 (2003); Zheng et al., Angew. Chem., Int. Ed. 48, 7626-7629 (2009); Giam et al., Proc. Natl. Acad. Sci. U. S. A. 109, 4377-4382 (2012); Slater et al., Micropatterning in Cell Biology, Pt A 119, 193-217 (2014); Wang et al., Nano Lett. 15, 1457-1467 (2015)]. Importantly, the use of approaches to pattern adhesive cues has provided significant insight into more complex behaviors including migration [Boujemaa-Paterski et al., Methods Enzymol. 540, 283-300 (2014); Thery et al., Proc. Natl. Acad. Sci. U. S. A. 103, 19771-19776 (2006)], differentiation [Kilian et al., Proc. Natl. Acad. Sci. U. S. A. 107, 4872-4877 (2010); Shukla et al., ACS Appl. Mater. Interfaces 8, 21883-21892 (2016); Dike et al., In Vitro Cell. Dev. Biol. Anim. 35, 441-448 (1999); von Erlach et al., Nat. Mater. 17, 237-+(2018); McBeath et al., Dev Cell 6, 483-495 (2004)], survival [Dike et al., In Vitro Cell. Dev. Biol. Anim. 35, 441-448 (1999); Thery et al., Nat. Cell Biol. 7, 947-953 (2005); Chen et al., Science 276, 1425-1428 (1997)], and signaling [Jain et al., Proc. Natl. Acad. Sci. U.S.A. 110, 11349-11354 (2013); Spatz et al., Methods Cell Biol. 83, 89-111 (2007); Tseng et al., Proc. Natl. Acad. Sci. U.S.A. 109, 1506-1511 (2012)]. Significant work has used microcontact printing to demonstrate how global cell shape directs the formation of focal adhesions at local regions of the cell perimeter [Petty et al., J. Am. Chem. Soc. 129, 8966-+(2007); Thery et al., J. Cell Sci. 123, 4201-4213 (2010); Vignaud et al., J. Cell Sci. 125, 2134-2140 (2012)]. It is also commonly observed that FAs adopt an elongated morphology at edges of the cell, with an alignment that corresponds with that of the associated actin stress filaments.

A series of landmark studies have demonstrated how substrates that are nanopatterned with ECM ligands can control cell adhesion and influence cell activities. Spatz and coworkers, for example, used micelle block-copolymer lithography to show that a maximal distance of approximately 50-70 nm between individual integrin molecules can still support integrin clustering for effective cell adhesion and spreading, and where greater inter-ligand spacing decreases cell function [Arnold et al., Nano Lett. 8, 2063-2069 (2008)]. Furthermore, nanostructured topographies presenting cell adhesion ligands can enhance osteogenic differentiation when moderate disorder is present, which supports the formation of stable focal adhesions [Huang et al., Nano Lett. 9, 1111-1116 (2009); Dalby et al., Nat. Mater. 6, 997 (2007)]. Other studies further reveal the exquisite sensing capability of cells to respond to nanoscale variations in the size, spacing, composition and topology of patterned cues presented on substrates [Geiger et al., Nat. Rev. Mol. Cell Biol. 10, 21-33 (2009); Lohmuller et al., Biointerphases 6, MR1-12 (2011); Arnold et al., Nano Lett. 8, 2063-2069 (2008); Cavalcanti-Adam et al., HFSP J. 2, 276-285 (2008); Selhuber-Unkel et al., Biophys. J. 98, 543-551 (2010); Spatz et al., Methods Cell Biol. 83, 89-111 (2007); Arnold et al., Soft Matter 5, 72-77 (2009); McMurray et al., Nat. Mater. 10, 637-644 (2011)].

### SUMMARY

In some aspects, the present disclosure provides a method of making an oriented array, comprising printing a surface with an array of a cell adhesion ligand by (i) coating a polymer pen lithography (PPL) tip array with a monolayer reagent, (ii) printing the monolayer reagent at selected positions on the surface to form an array having a selected orientation of printed monolayer reagent, (iii) contacting the array of printed monolayer reagent with the cell adhesion ligand under conditions to immobilize the cell adhesion ligand to the surface at the printed monolayer reagent positions from step (ii), thereby forming the oriented array of the cell adhesion ligand. In some embodiments, the monolayer reagent is mercaptohexadecanoic acid (MHA). In some embodiments, the cell adhesion ligand is a protein, a peptide, or an antibody. In further embodiments, the protein is an extracellular matrix (ECM) protein. In some embodiments, the ECM protein is fibronectin, collagen, elastin, vitronectin, bone sialoprotein, or laminin. In some embodiments, the peptide is an RGD peptide, a GFOGER (SEQ ID NO: 1) peptide, and/or a YGISR (SEQ ID NO: 1) peptide.

In some embodiments, a method of the disclosure further comprises (iv) introducing a second monolayer that is a bio-inert region that inhibits association with the cell.

In some embodiments, the surface comprises a multi-well plate. In further embodiments, the surface comprises gold, silver, silica, glass, quartz, a metal-oxide, or copper. In some embodiments, the metal-oxide is TiO₂, AlO₃, or ItO).

In some embodiments, more than one cell is contacted with the surface. In further embodiments, 2, 5, 10, 20, 50, or 100 cells are contacted with the surface.

In some embodiments, the cell adhesion ligand is bound to the surface via a linker. In further embodiments, the linker has a structure of formula I:

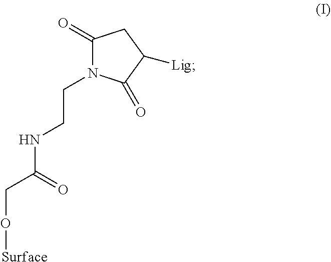

where Lig comprises the cell adhesion ligand.

In some embodiments, the surface comprises a monolayer. In further embodiments, the monolayer comprises (i) the linker and (ii) an ethylene glycol and a C₂₋₂₀ alkylene moiety. In some embodiments, the monolayer is attached to the surface via a thiol bond.

In some embodiments, the PPL tip array comprises a compressible elastomeric polymer comprising a plurality of non-cantilevered tips each having a radius of curvature of less than 1 μm and a common substrate comprising a compressible elastomeric polymer, the tip array and the common substrate mounted onto a rigid support and the tip array, common substrate, and rigid support together being at least translucent. In further embodiments, the compressible elastomeric polymer comprises polydimethylsiloxane (PDMS).

In some aspects, the disclosure provides a patterned array produced by any of the methods disclosed herein.

In some aspects, a method of modulating cytoskeletal formation in a cell is provided, comprising providing a nanoscale pattern of a cell adhesion ligand on a surface by (i) coating a polymer pen lithography (PPL) tip array with a monolayer reagent, (ii) printing the monolayer reagent at selected positions on the surface to form an array of a selected orientation of printed monolayer reagent, (iii) contacting the array of printed monolayer reagent with the cell adhesion ligand under conditions to immobilize the cell adhesion ligand to the surface at the printed monolayer reagent positions from step (ii), thereby forming an oriented array of the cell adhesion ligand; contacting the surface with the cell and then culturing the cell to allow for cell growth; wherein orientation of the array of the cell adhesion ligand modulates cytoskeletal formation in the cell. In some embodiments, the method further comprises (iv) introducing a second monolayer that is a bio-inert region that inhibits association with the cell. In some embodiments, orientation of the array promotes uniform cell size and/or shape. In some embodiments, orientation of the array promotes differentiation of the cell. In further embodiments, the method further comprises contacting the cell with a growth and/or differentiation factor. In some embodiments, the growth factor is h-insulin, TGF-β, VEGF, IL-3, IL-6, IL-11, EGF, FGF, Oct-3, Sox2, BMP, IGF, Activin, Wnt, or a combination thereof. In further embodiments, the differentiation factor is dexamethasone, ascorbate, L-glutamine, B-glycerophosphate, indomethacin, 3-isobutyl-l-methyl-xanthine, or a combination thereof.

In some embodiments, the cell is a stem cell, cancer cells, a neuronally-derived cell, or a combination thereof. In various embodiments, the cell is any type of adherent cell. In further embodiments, the stem cell is a human mesenchymal stem cell (hMSC), a fibroblast, an induced-pluripotent stem cell (IPSO), an epidermal stem cell, a hemopoeitic stem cell, an embryonic stem cell, a neural stem cell, or a dermal stem cell.

### BRIEF DESCRIPTION OF FIGURES

FIG. 1 depicts a workflow for cell patterning and patterns utilized in cell patterning experiments. A) A workflow showing the process for creating patterns for cell adhesion. B) Pattern designs used in cell patterning experiments. C) Fluorescence micrographs of fibronectin on the patterned surfaces. See also FIG. 6.

FIG. 2 shows cytoskeletons of cells grown on patterns. a) Designs of patterns corresponding to each column. b) Computer representation of 3D structure of cells on patterns. c) Maximum intensity projection of a the actin fibers (red) and nuclei (blue) with a cell for each pattern. d) Actin fibers staining within a single cell. e) Heat map of images corresponding to the average intensity of actin fibers from maximum intensity projections of 45-50 micrographs for each pattern.

FIG. 3 shows a histogram of actin fiber orientations in hMSCs on square dot and anisotropic patterns. The anisotropic pattern shows preferential alignment along the 45 degree axis compared to a square dot pattern.

FIG. 4 shows: a) Map of the pixel orientations within a single cell. b) Pixels are classified based on whether they align along the radial or circumferential direction.

FIG. 5 shows: a-b) Micrograph of the actin fibers within a single cell (left) and heat maps of 50 cells averaged together (right). c-d) Histogram of pixels aligned along the radial axis as a function of angle around the cell. e-f) 1D FFT of the histograms for all cells.

FIG. 6 shows PPL patterning of cell attachment features. a, Workflow for patterning fibronectin features on a gold substrate for mediating cell attachment. b, Micrograph of an etched gold substrate visualizing the mercaptohexadecanoic acid (MHA) features. c, Fluorescence micrograph of antibody stained fibronectin that selectively adsorbed to the MHA patterns.

FIG. 7 shows actin fiber orientation within cells on square substrates. a-d, Pattern designs (left) and representative fluorescence micrographs of the actin cytoskeleton in hMSCs seeded on each pattern (right). e, Fluorescence image of fibronectin patterned as a square dot matrix (left), representative fluorescence image of the actin cytoskeleton of a cell on a square dot matrix pattern (center), heatmap of the actin fibers in cells on a square dot matrix substrate (n=78; right). f, Fluorescence image of fibronectin patterned as an anisotropic square (left), representative fluorescence image of the actin cytoskeleton of a cell on an anisotropic square pattern (center), heatmap of the actin fibers in cells on an anisotropic square substrate (n=64; right). g, The actin fiber orientation within cells on anisotropic and square dot matrix patterns. (mean±s.e.m.; **p<0.01, ***p<0.001, two-way ANOVA with Bonferroni post hoc).

FIG. 8 shows sets of patterns used to identify cytoskeletal controlling features in a square geometry. a-d, Computer generated images of the programmed fibronectin features patterned on gold substrates and representative fluorescence images of the actin cytoskeleton (red) for cells on these patterns are shown along with the nucleus (blue).

FIG. 9 shows sets of patterns used to identify cytoskeletal controlling features in a circular geometry. a-h, Computer generated images of the programmed fibronectin features patterned on gold substrates and representative fluorescence images of the actin cytoskeleton (red) for cells on these patterns are shown along with the nucleus (blue).

FIG. 10 shows actin fiber orientation within cells on circular substrate. a, Fluorescence micrograph of fibronectin patterned as a dot matrix circle. b, Representative fluorescence images of the actin cytoskeleton of cells seeded on dot matrix circle patterns. c, Heatmap of the actin fibers in cells on dot matrix circles (n=84). d, Fluorescence micrograph of fibronectin patterned as a 20-point circle. e, Representative images of the actin cytoskeleton of cells seeded on 20-point circles. f, Heatmap of the actin fibers in cells on the 20-point circle (n=57). g, The angular distribution of radial fibers within the cells on dot matrix and 20-point circle patterns (mean±s.e.m.). h, FFT of the angular distribution of the radially oriented actin fibers (mean±s.d.).

FIG. 11 shows fiber classification within cells on circular geometries. a, Local fiber orientation of all fibers within the cell. b, Classification of fiber directionality within the cell with respect to the centroid.

FIG. 12 shows fluorescence micrographs of the focal adhesions and actin cytoskeleton within cells on patterns. a-d, Representative confocal images of single cells grown on different patterns: square dot matrix (a), anisotropic square (b), dot matrix circles (c), and 20-point circles (d). The nucleus (Panel 1), focal adhesions (vinculin, panel 2), and actin cytoskeleton (panel 3) are labeled within each cell. Panel 4 shows the overlay of the different structures.

FIG. 13 shows osteogenic and adipogenic differentiation on square patterns. a, Examples of cells on each respective pattern staining purple as an osteogenic marker (left column) and red as an adipogenic marker (right column). Scale bar: 25μm. b, Cumulative distributions staining intensity after color deconvolution of the red and purple channels from 3 substrates (n_{cells}=759-817; ***p<0.001; **p<0.01; *p<0.05; Mann-Whitney U). c, Percentage of cells staining positive for only adipogenic or osteogenic markers (mean ±s.e.m.; *** p<0.001; *p<0.05; one-way ANOVA with Tukey HSD).

FIG. 14 shows osteogenic and adipogenic differentiation on circular patterns. a, Examples of cells on each respective pattern staining purple as an osteogenic marker (left column) and red as an adipogenic marker (right column). Scale bar: 25μm. b, Cumulative distributions of staining intensity after color deconvolution of the red and purple channels from 3 substrates (n_{cellss}=754-849; ***p<0.001; * p<0.05; Mann-Whitney U). c, Percentage of cells staining positive for only adipogenic or osteogenic markers (mean±s.e.m.; * p<0.0246; t-Test).

FIG. 15 shows color deconvolution of cells stained for alkaline phosphatase and lipid vacuoles. Top: The purple channel and red channel intensity for a cell staining positive for osteogenesis. Bottom: The purple channel and red channel intensity for a cell staining positive for adipogenesis.

FIG. 16 shows fluorescence micrographs of myosin IIA within cells on patterns. a-d, Additional confocal images of single cells grown on different patterns: square dot matrix (a), anisotropic square (b), dot matrix circles (c), and 20-point circles (d). The nucleus (Panel 1), myosin Ila (Panel 2), and vinculin (focal adhesions) (Panel 3) are labeled within each cell. Panel 4 shows the overlay of the different structures.

### DETAILED DESCRIPTION

The cytoskeleton is a complex and dynamic network of filaments that support cellular functions and maintain homeostasis. Rearrangement of the cell cytoskeleton is a ubiquitous process that occurs during important cell functions, such as adhesion, migration, division, differentiation, among others.¹⁻² These processes are mediated, in part, by specific interactions between cell adhesion proteins and the extracellular matrix (ECM), which result in the formation of focal adhesion complexes that physically link the actin filament network within the cell to the ECM, and more importantly, regulate signaling transduction pathways.³⁻⁴ Understanding how to control the arrangement of the actin cytoskeleton with molecular precision allows one to reduce the inherent heterogeneity present in cell cultures, which would enable cellular programming and improve cell-based assays having a more homogenous cell population. Achieving this goal remains challenging because cell matrix remodeling occurs even when the cell is confined to a patterned shape and it is unclear how sub-cellular control over the arrangement and distribution of ECM adhesion cues presented to the cell leads to control over the spatial distribution of focal adhesions and subsequent assembly of actin stress fibers following a user-defined orientation.

Exquisite control over actin network organization can be achieved by engineering substrates that directly present highly organized ECM cues to engage integrin cell surface receptors at desired locations. To this end, micro-and nano-patterning approaches—i.e. micro-contact printing,⁵⁻⁶ laser ablation lithography,^{∂}and micelle nanolithography⁸⁻¹¹—have been used to template the arrangement of adhesion ligands to control cell shape,¹² geometry,¹³ and contractilityl⁴⁻¹⁵ and manipulate cellular processes, such as cell adhesion, migration, and differentiation.¹⁶⁻¹⁷ Studies utilizing nanopatterned substrates have elucidated the role of nanoscale ordering of cell adhesion peptides (e.g., RGD ligands) in activating integrin clustering and modulating cell adhesion.¹⁰ These studies revealed that the size and shape focal adhesions depends on the density and size of adhesive ligand with mature focal adhesions having dimensions in the order of micrometers and exhibiting an elongated morphology.^{10, 18}

Micro- and nano-patterning have been further extended to study how shape, ligand arrangement, and feature size affect cell differentiation.^{5, 19} It has been shown that micropatterned substrates can be used to define cell shape, characterize the relationship the interplay of cytoskeletal elements, and program human mesenchymal stem cell (hMSC) differentiation to desired fates.⁶ These studies have revealed that cytoskeletal contractility directly influences cell differentiation.⁵ Previous studies have used patterned substrates wherein the presentation of ligands is found in a symmetric feature (typically, a circle). Studies did not explore the role of feature anisotropy in directing cell behavior, in large part because the patterning methods to control geometry at a sub-micron length scale were not readily available. Indeed, most high-resolution nanopatterning techniques are labor intensive, time consuming, and provide limited throughput.

In the present disclosure, however, a high throughput, high resolution, large area nanopatterning technique, polymer pen lithography (PPL) [Eichelsdoerfer et al., Nat. Protoc. 8, 2548-2560 (2013); Huo et al., Science 321, 1658-1660 (2008); Zheng et al., Angewandte Chemie International Edition 48, 7626-7629 (2009); Wong et al., Nanoscale 4, 659-666 (2012)] was used to pattern sub-micron fibronectin features with aspect ratios of 4-6:1 and it is shown herein that the orientation of the fibronectin feature directed the alignment of the actin stress filaments in adherent cells [Giam et al., Proc. Natl. Acad. Sci. U.S.A. 109, 4377-4382 (2012); Eichelsdoerfer et al., Nat. Protoc. 8, 2548-2560 (2013); Huo et al., Science 321, 1658-1660 (2008); Cabezas et al., Methods Cell Biol. 119, 261-276 (2014); Cabezas et al., Nano Lett. 17, 1373-1377 (2017)]. PPL can be used to define subcellular protein features in arbitrary size and shape over cm² areas, allowing one to probe many cells at once under near-identical conditions, thereby providing a route to statistically meaningful data. The present disclosure describes this technique, and by way of example, demonstrates the technique to nanopattern fibronectin into pre-defined geometries and the subcellular arrangements to test the influence of anisotropy as a cue that directs the assembly of the cytoskeleton and downstream signaling. As shown in the Examples below, anisotropic focal adhesions provided control over the uniformity and directionality of the actin cytoskeleton. While it has been shown that the anisotropy of focal adhesions increases with increasing cell aspect ratios [Ray et al., Nat. Commun. 8 (2017)], the use of PPL allows independent control over cell shape and anisotropic presentation of fibronectin and therefore provides an understanding of how sub-cellular cues can independently direct assembly of the cytoskeleton with control over contractility and differentiation. The present disclosure demonstrates how this approach has identified two new shape factors that enhance osteogenesis.

The present disclosure therefore relates to nanolithographical cell patterning. In some aspects, the disclosure provides materials and methods for improving cell uniformity for cell-based assays used in drug discovery and enhancing cell differentiation for tissue engineering. The present disclosure specifically addresses the spatial arrangement of adhesion cues within a defined geometry with sub-cellular control to direct the assembly and distribution of focal adhesions. Furthermore, it is disclosed herein that the organization and direction of actin filaments within cells can be carefully controlled by presenting patterned geometries of ECM proteins (such as fibronectin) having individual nano-sized ECM features distributed and oriented along the periphery of a geometric shape such that integrin clustering and formation of focal adhesions induces actin filaments to follow the direction of the underlying pattern.

To impart organization and directionality to the actin cytoskeleton, a cantilever-free scanning probe lithographic technique, termed polymer pen lithography (PPL), was used. This massively parallel technique utilizes an elastomeric pen array to directly deliver materials (e.g. alkanethiols, DNA, proteins) to substrates.²⁰ Customizable patterns having feature sizes ranging from the nano-to microscale are easily generated by simply changing either the amount of force applied to the pen array or the tip-substrate dwell time. This mask-free and high-throughput technique is uniquely positioned to systematically generate user-defined structures that enable synthesis of combinatorial libraries for cell adhesion studies and cell-based assays with molecular readouts.^{19, 21-22} In this work, PPL was used to pattern self-assembled monolayers (SAMs) of alkanethiolates on gold that present fibronectin and control the shape and sizes of single cells in culture. The approach disclosed herein utilizes PPL to rapidly prototype nanopatterns of ECM proteins that induce orientation of actin stress fibers and direct stem cell fate. Significantly, it is demonstrated herein that arbitrary ECM pattern arrangements are easily explored and small modifications in the pattern are used to modulate cytoskeletal organization and promote osteogenic stem cell fate.

Cells have unique shapes that play significant role in their function. Nano- and micro-patterning approaches have been used to control cell behavior, location, and fate by chemically defining interactions between cells and surfaces. These techniques have been used to identify and create specific shapes that influence basic cellular processes such as survival and differentiation and to spatially confine cells to desired locations on substrates for cell-based assays. The use of specific cell shapes can alter the arrangement and contractility of the cell's cytoskeleton by defining the formation of focal adhesions and stress fibers. Overall, these external cues are transduced into biochemical signals, which modulate signaling pathways within the cell, ultimately altering cell fate. While refined control over the arrangement of actin fibers around the periphery of the cell has been achieved, the arrangement of actin fibers/bundles within the cells remains largely uncontrolled. Achieving higher control over the cytoskeleton has the potential to enhance desired cell outcomes (e.g., differentiation, polarization, enzymatic activity) and also create a more homogenous cell population for use in cell assays. Here, nanopatterning is utilized to spatially define specific locations where cells can interact with a substrate to form a focal adhesion, a specific interaction that couples the actin cytoskeleton to the surrounding environment. By arranging the focal adhesions in unique patterns, actin fibers within a cell are oriented in a desired direction. This increased control over the actin cytoskeleton provides better control over cell differentiation and enhances the uniformity of the cells on a substrate.

Applications of the technology disclosed herein include, but are not limited to, cell-based screening for drug discovery and differentiation of stem cells to specific targeted lineages for tissue engineering. The present disclosure provides, in various aspects, a high throughput approach to patterning cells with greater uniformity as well as better control over cell contractility.

Self-Assembled Monolayer Surfaces. The present disclosure contemplates the use of self-assembled monolayers as surfaces for various applications (Mrksich et al., Annu Rev Biophys Biomol Struct 25: 55-78 (1996); Hodneland et al., Langmuir 13: 6001-6003 (1997); Houseman et al., FASEB J 11: A1095-A1095 (1997); Mrksich, Curr Opin Colloid In 2: 83-88 (1997); Mrksich et al., Acs Sym Ser 680: 361-373 (1997); Houseman et al., Mol Biol Cell 9: 430a-430a (1998); Mrksich, Cell Mol Life Sci 54: 653-662 (1998); Houseman et al., Angew Chem Int Ed 38: 782-785 (1999); Li et al., Langmuir 15: 4957-4959 (1999); Yousaf et al., J Am Chem Soc 121: 4286-4287 (1999); Houseman et al., Mol Biol Cell 11: 45a-45a (2000); Luk et al., Langmuir 16: 9604-9608. (2000); Mrksich, Chem Soc Rev 29: 267-273 (2000); Yousaf et al., Angew Chem Int Ed Engl 39: 1943-1946 (2000); Yousaf et al., Biochemistry 39: 1580-1580 (2000); Houseman et al., Biomaterials 22: 943-955 (2001); Kato et al., Biochemistry 40: 8608-8608 (2001); Yeo et al., Chembiochem 2: 590-593 (2001); Yousaf et al., Proc Natl Acad Sci USA 98: 5992-5996. (2001); Yousaf et al., Angew Chem Int Ed Engl 40: 1093-1096 (2001); Hodneland et al., Proc Natl Acad Sci USA 99: 5048-5052 (2002); Houseman et al., Nat Biotechnol 20: 270-274 (2002); Houseman et al., Top Curr Chem 218: 1-44 (2002); Houseman et al., Trends Biotechnol 20: 279-281 (2002); Houseman et al., Chem Biol 9: 443-454 (2002); Kwon et al., J Am Chem Soc 124: 806-812 (2002); Lee et al., Science 295: 1702-1705 (2002); Mrksich, Curr Opin Chem Biol 6: 794-797 (2002); Houseman et al., Langmuir 19: 1522-1531 (2003); Luk et al., Biochemistry 42: 8647-8647 (2003); Yeo et al., Angew Chem Int Ed Engl 42: 3121-3124 (2003); Dillmore et al., Langmuir 20: 7223-7231 (2004); Feng et al., Biochemistry 43: 1 581 1-1 5821 (2004); Kato et al., J Am Chem Soc 126: 6504-6505 (2004); Min et al., Curr Opin Chem Biol 8: 554-558 (2004); Murphy et al., Langmuir 20: 1026-1030 (2004); Yeo et al., Adv Mater 16: 1352-1356 (2004); Yonzon et al., J Am Chem Soc 126: 12669-12676 (2004); Mrksich, MRS Bull 30: 180-184 (2005); James et al., Cell Motil Cytoskeleton 65: 841-852 (2008)). The monolayers offer the benefits that immobilized ligands are presented and can be patterned in a homogeneous environment and the density of the immobilized ligands can be controlled and made uniform across the entire patterned array (Gawalt et al., J Am Chem Soc 126: 15613-7 (2004)). The monolayers are also compatible with a range of immobilization chemistries (Montavon et al., Nat Chem 4: 45-51 (2012); Ban et al., Nat Chem Biol 8: 769-773 (2012); Li et al., Langmuir 23, 11826-11835 (2007)). A significant benefit of the monolayer substrates is that they can be analyzed by matrix-assisted laser desorption-ionization mass spectrometry (i.e., SAMDI mass spectrometry) and therefore provide a route to label-free assays of biochemical activities (Su et al., Langmuir 19: 4867-4870 (2003)).

Monolayer Reagent. The monolayer on the surface is prepared in two steps—(1) patterning a first monolayer reagent onto selection sections of the surface then (2) incubating the second monolayer reagent so as to adhere to the unpatterned sections of the surface—thereby creating a surface covered in monolayer reagent(s). The first monolayer can be the reagent that is capable of adsorption of the cell adhesion ligand. The second monolayer reagent is the reagent not used in the first (PPL patterning) step. These steps may be performed in any order. For example and without limitation, one can pattern nonadherent regions first and then backfill with a monolayer that promotes cell adhesion.

In some embodiments, a monolayer reagent capable of adsorption of the cell adhesion ligand is a monolayer reagent for nonspecific adsorption of protein. In some cases, such a monolayer reagent has a structure of HS(CH₂)ₙX, where n is 8-20 and X is methyl, OH, OC₁₋₃alkyl, CO₂H, or NH₂. More specific examples of such monolayer reagents include mercaptohexadecanoic acid (MHA), a C₈₋₂₀ hydroxyalkane, and hexadecane thiolate.

The monolayer reagent can be bound to the surface via a thiol bond (e.g., the monolayer reagent comprises a thiol—SH—and reacts with the surface to form a bond via the sulfur atom). In some cases, the monolayer reagent can further comprise a linker. For example, the linker can have a structure of formula (I)

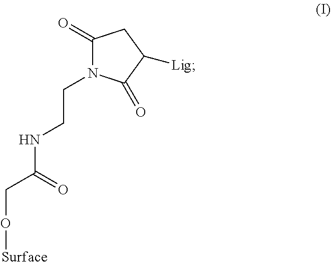

where Lig comprises the cell adhesion ligand.

Surface. The surface can be any material capable of forming a monolayer, e.g., a monolayer of alkanethiols. Particularly, the surface may be a metal, such as Au, Ag, Pd, Pt, Cu, Zn, Fe, In, Si, Fe₂O₃, SiO₂ or ITO (indium tin oxide) glass. In various embodiments, the disclosure contemplates that a surface useful in the methods described herein comprises Au, Ag, or Cu. In some cases, the surface comprises Au.

Surfaces suitable for use in methods disclosed herein include, but are not limited to, metals, alloys, composites, crystalline materials, amorphous materials, conductors, semiconductors, optics, fibers, inorganic materials, glasses, ceramics (e.g., metal oxides, metal nitrides, metal silicides, and combinations thereof), zeolites, polymers, plastics, organic materials, minerals, biomaterials, living tissue, bone, films thereof, thin films thereof, laminates thereof, foils thereof, composites thereof, and combinations thereof. A surface can comprise a semiconductor such as, but not limited to: crystalline silicon, polycrystalline silicon, amorphous silicon, p-doped silicon, n-doped silicon, silicon oxide, silicon germanium, germanium, gallium arsenide, gallium arsenide phosphide, indium tin oxide, and combinations thereof. A surface can comprise a glass such as, but not limited to, undoped silica glass (SiO₂), fluorinated silica glass, borosilicate glass, borophosphorosilicate glass, organosilicate glass, porous organosilicate glass, and combinations thereof. The surface can be a non-planar surface, such as pyrolytic carbon, reinforced carbon-carbon composite, a carbon phenolic resin, and the like, and combinations thereof. A surface can comprise a ceramic such as, but not limited to, silicon carbide, hydrogenated silicon carbide, silicon nitride, silicon carbonitride, silicon oxynitride, silicon oxycarbide, high-temperature reusable surface insulation, fibrous refractory composite insulation tiles, toughened unipiece fibrous insulation, low-temperature reusable surface insulation, advanced reusable surface insulation, and combinations thereof. A surface can comprise a flexible material, such as, but not limited to: a plastic, a metal, a composite thereof, a laminate thereof, a thin film thereof, a foil thereof, and combinations thereof.

Cell adhesion ligand. As discussed herein, aspects of the disclosure contemplate the use of a surface comprising an immobilized cell adhesion ligand. In any of the aspects or embodiments of the disclosure, the surface further comprises a bio-inert region. A bio-inert region as used herein is a region that repels cells or otherwise inhibits attachment of a cell. In some embodiments, the bio-inert region comprises a ligand that repels a cell. In various embodiments, the ligand that repels a cell is a monolayer terminated in ethylene glycol, a fluorinated group, heparin, or a tannic acid.

Cell adhesion ligands contemplated by the disclosure include any ligand that helps a cell attach to a surface or to another cell. In various embodiments, the cell adhesion ligand is a protein, a peptide, an antibody, an aptamer, or a combination thereof. In some embodiments, the cell adhesion ligand comprises an amino acid sequence such as, for example and without limitation, RGD or GRTY (SEQ ID NO: 3). In some embodiments, the cell adhesion ligand comprises an extracellular matrix (ECM) protein, including but not limited to fibronectin, collagen, elastin, vitronectin, bone sialoprotein, and/or laminin. In some embodiments, the cell adhesion ligand is an antibody that specifically binds an integrin protein.

Multiplexing. The methods of the disclosure are amenable to the multiplex format. Thus, in any of the aspects or embodiments of the disclosure, simultaneous exposure of a cell on a surface to more than one, e.g., cell adhesion ligand or growth factor is contemplated.

Polymer Pen Lithography (PPL). Polymer Pen Lithography is generally disclosed in International Application No. PCT/US09/041738 (WO 09/132321), and is a direct-write method that delivers collections of molecules in a positive printing mode. In contrast with DPN and other SPM-based lithographies, which typically use hard silicon-based cantilevers, Polymer Pen Lithography utilizes elastomeric tips without cantilevers) as the ink delivery tool. The tips are preferably made of polydimethylsiloxane, PDMS. A preferred polymer pen array contains thousands of tips, preferably having a pyramidal shape, which can be made with a master prepared by conventional photolithography and subsequent wet chemical etching. The tips preferably are connected by a common substrate which includes a thin polymer backing layer (50-100 μm thick), which preferably is adhered to a rigid support (e.g., a glass, silicon, quartz, ceramic, polymer, or any combination thereof), e.g. prior to or via curing of the polymer. The rigid support is preferably highly rigid and has a highly planar surface upon which to mount the array (e.g., silica glass, quartz, and the like). The rigid support and thin backing layer significantly improve the uniformity of the polymer pen array over large areas, such as three inch wafer surface, and make possible the leveling and uniform, controlled use of the array. When the sharp tips of the polymer pens are brought in contact with a substrate, ink is delivered at the points of contact.

The amount of light reflected from the internal surfaces of the tips increases significantly when the tips make contact with the substrate. Therefore, a translucent or transparent elastomer polymer pen array allows one to visually determine when all of the tips are in contact with an underlying substrate, permitting one to address the otherwise daunting task of leveling the array in an experimentally straightforward manner. Thus, preferably one or more of the array tips, backing layer, and rigid support are at least translucent, and preferably transparent.

Depending upon intended use, the pitch of a pen array is deliberately set between 20 μm and 1 mm, corresponding to pen densities of 250,000/cm² and 100/cm², respectively. Larger pitch arrays are required to make large features (micron or millimeter scale) but also can be used to make nanometer scale features. All of the pens were remarkably uniform in size and shape, with an average tip radius of 70±10 nm. In principle, this value can be reduced substantially with higher quality masters and stiffer elastomers. For the examples below, the tip array used contained either 15,000 or 28,000 pyramid-shaped pens, but arrays with as many as about 11,000,000 pens have also been used to pattern structures.

In a typical experiment, a pen array (1 cm² in size) can be inked by immersing it in a saturated solution of a desired material, e.g., 16-mercaptohexadecanoic acid (MHA) in ethanol, for five minutes followed by rinsing, e.g., with ethanol. The inked pen array can be used for to generate patterns on a substrate by bringing it in contact with the surface for a period of time (e.g., 0.1 s). This process of contacting the substrate can be repeated to generate an array of patterns (e.g., dots) with less than 10% deviation in feature diameter.

A defining characteristic of Polymer Pen Lithography, in contrast with DPN and most contact printing strategies which are typically viewed as pressure or force-independent, is that it exhibits both time- and pressure-dependent ink transport. As with DPN, features made by Polymer Pen Lithography exhibit a size that is linearly dependent on the square root of the tip-substrate contact time. This property of Polymer Pen Lithography, which is a result of the diffusive characteristics of the ink and the small size of the delivery tips, allows one to pattern sub-micron features with high precision and reproducibility (variation of feature size is less than 10% under the same experimental conditions). The pressure dependence of Polymer Pen Lithography derives from the compressible nature of the elastomer pyramid array. Indeed, the microscopic, preferably pyramidal, tips can be made to deform with successively increasing amounts of applied pressure, which can be controlled by simply extending the piezo in the vertical direction (z-piezo). Although such deformation has been regarded as a major drawback in contact printing (it can result in “roof” collapse and limit feature size resolution), with Polymer Pen Lithography, the controlled deformation can be used as an adjustable variable, allowing one to control tip-substrate contact area and resulting feature size. Within the pressure range allowed by z-piezo extension of about 5 to about 25 μm, one can observe a near linear relationship between piezo extension and feature size at a fixed contact time of 1 s. Interestingly, at the point of initial contact and up to a relative extension 0.5 μm, the sizes of the patterned dots do not significantly differ and are both about 500 nm, indicating that the backing elastomer layer, which connects all of the pyramids, deforms before the pyramid-shaped tips do. This type of buffering is fortuitous and essential for leveling because it provides extra tolerance in bringing all of the tips in contact with the surface without tip deformation and significantly changing the intended feature size. When the z-piezo extends 1 μm or more, the tips exhibit a significant and controllable deformation.

With the pressure dependency of Polymer Pen Lithography, one does not have to rely on the time-consuming, meniscus-mediated ink diffusion process to generate large features. Indeed, one can generate either nanometer or micrometer sized features in only one printing cycle by simply adjusting the degree of tip deformation.

Note that the maskless nature of Polymer Pen Lithography allows one to arbitrarily make many types of structures without the hurdle of designing a new master via a throughput-impeded serial process.

Polymer Pen Lithography merges many of the attributes of DPN and contact printing to yield patterning capabilities that span multiple length scales with high throughput and low cost. The time- and pressure-dependent ink transport properties of the polymer pen pyramid arrays provide important and tunable variables that distinguish Polymer Pen Lithography from the many nano- and microfabrication approaches that have been developed to date.

Tip Arrays. The lithography methods disclosed herein employ a tip array formed from elastomeric polymer material. The tip arrays are non-cantilevered and comprise tips which can be designed to have any shape or spacing between them, as needed. The shape of each tip can be the same or different from other tips of the array. Contemplated tip shapes include spheroid, hemispheroid, toroid, polyhedron, cone, cylinder, and pyramid (trigonal or square). The tips are sharp, so that they are suitable for forming submicron patterns, e.g., less than about 500 nm. The sharpness of the tip is measured by its radius of curvature, and the radius of curvature of the tips disclosed herein is below 1 μ, and can be less than about 0.9 μm, less than about 0.8 μm, less than about 0.7 μm, less than about 0.6 μm, less than about 0.5 μm, less than about 0.4 μm, less than about 0.3 μm, less than about 0.2 μm, less than about 0.1 μm, less than about 90 nm, less than about 80 nm, less than about 70 nm, less than about 60 nm, or less than about 50 nm.

The tip array can be formed from a mold made using photolithography methods, which is then used to fashion the tip array using a polymer as disclosed herein. The mold can be engineered to contain as many tips arrayed in any fashion desired. The tips of the tip array can be any number desired, and contemplated numbers of tips include about 1000 tips to about 15 million tips, or greater. The number of tips of the tip array can be greater than about 1 million, greater than about 2 million, greater than about 3 million, greater than about 4 million, greater than 5 million tips, greater than 6 million, greater than 7 million, greater than 8 million, greater than 9 million, greater than 10 million, greater than 11 million, greater than 12 million, greater than 13 million, greater than 14 million, or greater than 15 million tips.

The tips of the tip array can be designed to have any desired thickness, but typically the thickness of the tip array is about 50 nm to about 1 μm, about 50 nm to about 500 nm, about 50 nm to about 400 nm, about 50 nm to about 300 nm, about 50 nm to about 200 nm, or about 50 nm to about 100 nm.

The polymers can be any polymer having a compressibility compatible with the lithographic methods. Polymeric materials suitable for use in the tip array can have linear or branched backbones, and can be crosslinked or non-crosslinked, depending upon the particular polymer and the degree of compressibility desired for the tip. Cross-linkers refer to multi-functional monomers capable of forming two or more covalent bonds between polymer molecules. Non-limiting examples of cross-linkers include such as trimethylolpropane trimethacrylate (TMPTMA), divinylbenzene, di-epoxies, tri-epoxies, tetra-epoxies, di-vinyl ethers, tri-vinyl ethers, tetra-vinyl ethers, and combinations thereof.

Thermoplastic or thermosetting polymers can be used, as can crosslinked elastomers. In general, the polymers can be porous and/or amorphous. A variety of elastomeric polymeric materials are contemplated, including polymers of the general classes of silicone polymers and epoxy polymers. Polymers having low glass transition temperatures such as, for example, below 25° C. or more preferably below −50° C., can be used. Diglycidyl ethers of bisphenol A can be used, in addition to compounds based on aromatic amine, triazine, and cycloaliphatic backbones. Another example includes Novolac polymers. Other contemplated elastomeric polymers include methylchlorosilanes, ethylchlorosilanes, and phenylchlorosilanes, polydimethylsiloxane (PDMS). Other materials include polyethylene, polystyrene, polybutadiene, polyurethane, polyisoprene, polyacrylic rubber, fluorosilicone rubber, and fluoroelastomers.

Further examples of suitable polymers that may be used to form a tip can be found in U.S. Pat. No. 5,776,748; U.S. Pat. No. 6,596,346; and U.S. Pat. No. 6,500,549, each of which is hereby incorporated by reference in its entirety. Other suitable polymers include those disclosed by He et al., Langmuir 2003, 19, 6982-6986; Donzel et al., Adv. Mater. 2001, 13, 1164-1167; and Martin et al., Langmuir, 1998, 14-15, 3791-3795. Hydrophobic polymers such as polydimethylsiloxane can be modified either chemically or physically by, for example, exposure to a solution of a strong oxidizer or to an oxygen plasma.

The polymer of the tip array has a suitable compression modulus and surface hardness to prevent collapse of the polymer during inking and printing, but too high a modulus and too great a surface hardness can lead to a brittle material that cannot adapt and conform to a substrate surface during printing. As disclosed in Schmid, et al., Macromolecules, 33:3042 (2000), vinyl and hydrosilane prepolymers can be tailored to provide polymers of different modulus and surface hardness. Thus, in some cases, the polymer is a mixture of vinyl and hydrosilane prepolymers, where the weight ratio of vinyl prepolymer to hydrosilane crosslinker is about 5:1 to about 20:1, about 7:1 to about 15:1, or about 8:1 to about 12:1.

The polymers of the tip array preferably will have a surface hardness of about 0.2% to about 3.5% of glass, as measured by resistance of a surface to penetration by a hard sphere with a diameter of 1 mm, compared to the resistance of a glass surface (as described in Schmid, et al., Macromolecules, 33:3042 (2000) at p 3044). The surface hardness can be about 0.3% to about 3.3%, about 0.4% to about 3.2%, about 0.5% to about 3.0%, or about 0.7% to about 2.7%. The polymers of the tip array can have a compression modulus of about 10 MPa to about 300 MPa. The tip array preferably comprises a compressible polymer which is Hookean under pressures of about 10 MPa to about 300 MPa. The linear relationship between pressure exerted on the tip array and the feature size allows for control of the indicia printed using the disclosed methods and tip arrays.

The tip array can comprise a polymer that has adsorption and/or absorption properties for the patterning composition, such that the tip array acts as its own patterning composition reservoir. For example, PDMS is known to adsorb patterning inks, see, e.g., US Patent Publication No. 2004/228962, Zhang, et al., Nano Lett. 4, 1649 (2004), and Wang et al., Langmuir 19, 8951 (2003).

The tip array can comprise a plurality of tips fixed to a common substrate and formed from a polymer as disclosed herein. The tips can be arranged randomly or in a regular periodic pattern (e.g., in columns and rows, in a circular pattern, or the like). The tips can all have the same shape or be constructed to have different shapes. The common substrate can comprise an elastomeric layer, which can comprise the same polymer that forms the tips of the tip array, or can comprise an elastomeric polymer that is different from that of the tip array. The elastomeric layer can have a thickness of about 50 μm to about 100μm. The tip array can be affixed or adhered to a rigid support (e.g., glass, such as a glass slide). In various cases, the common substrate, the tip array, and/or the rigid support, if present, is translucent or transparent. In a specific case, each is translucent or transparent. The thickness of combination of the tip array and common substrate, can be less than about 200 μm, preferably less than about 150 μm, or more preferably about 100 μm.

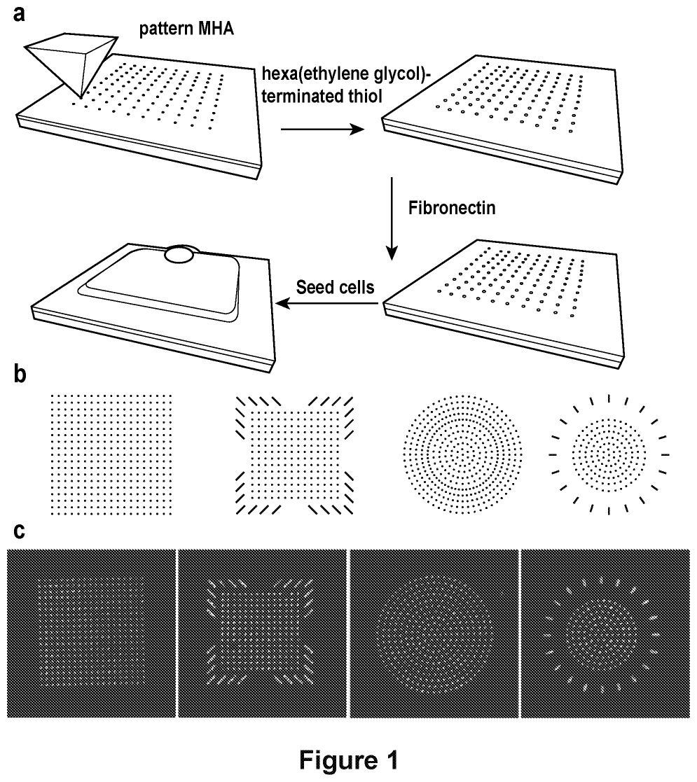

Printing of Surface. As disclosed herein a surface of the disclosure is printed with an array of a cell adhesion ligand. The array, in various embodiments, is printed in a selected orientation. Specifically, a polymer pen lithography (PPL) tip array is coated with a monolayer reagent, (ii) the monolayer reagent is printed at selected positions on the surface to form an array having a selected orientation of printed monolayer reagent, (iii) the array of printed monolayer reagent is contacted with the cell adhesion ligand under conditions to immobilize the cell adhesion ligand to the surface at the printed monolayer reagent positions from step (ii), thereby forming an oriented array of the cell adhesion ligand. The cell adhesion ligand is patterned such that it is clustered and forms in a particular orientation. Note that it is not the orientation of a particular cell adhesion ligand that is controlled, but rather the orientation of the cluster of cell adhesion ligands. See, e.g., FIG. 1. Controlling the orientation of the cell adhesion ligand provides benefits for application such as, without limitation, tissue engineering, drug discovery (screening therapeutics), and cell-based assays. The methods of the disclosure are also useful in situations where only few cells are available for assaying (e.g., from a patient sample).

The adhesion ligand pattern presented on the surface contributes to a particular outcome for a cell that is contacted with the surface. These outcomes include the ability to control cell uniformity. For example and without limitation, it is desirable in various assays for cells in various wells of a multiwell plate to express genes at a uniform level, and the cytoskeleton can help to control this. If the cell adhesion ligand is not patterned in a particular arrangement in the wells of the multiwell plate, then cells in different wells may attach to the surface in different ways and as a result not all cells will be equivalent.

In further applications of the disclosure, a cell or cells that is/are contacted with a surface comprising a patterned array of a cell adhesion ligand as described herein is/are differentiated into a particular cell type. Such methods, in various embodiments, further comprise contacting the cells with a growth factor to promote the differentiation. Growth factors contemplated by the disclosure include, but are not limited to one or more proteins such as h-insulin, TGF-β, VEGF, IL-3, IL-6, IL-11, EGF, FGF, Oct-3, Sox2, BMP, IGF, Activin, and/or Wnt. Cells may also be contacted with one or more chemical factors including dexamethasone, ascorbate, L-glutamine, B-glycerophosphate, indomethacin, and/or 3-isobutyl-l-methyl-xanthine. The cells will differentiate in the presence of the growth factor and in the absence of a particular oriented pattern of cell adhesion ligand, but having the oriented pattern of cell adhesion ligand promotes the uniform growth of the cells on the surface. This is advantageous because the oriented patterning of the cell adhesion ligand induces the vast majority of cells to react in a predictable manner (e.g., the majority of cells will differentiate into the desired cell type), whereas without the oriented pattern it would be expected that a much more non-uniform population of cells would result (e.g., fewer cells will differentiate into the desired cell type or more homogenous population of different call subtypes).

The present disclosure provides materials and methods for making an oriented array. The oriented array is useful, in various aspects and embodiments, for promoting uniform cell size and/or shape, or for differentiating a cell. The dimensions of the pattern can be defined according to the spreading characteristics of a specific cell type. Within a confined dimension and shape, the arrangement of adhesion ligands can be easily defined by the user. The use of PPL as disclosed herein enables printing virtually any type of arrangement. These arrangements can vary in length, density of ligand, size, and composition. By way of example, in the case of mesenchymal stem cells, arrangements that modulate contractility induce differentiation between adipogenic (fat) or osteogenic (bone) fates. Specifically, if these cells are stretched out, they are experiencing a highly contractile environment leading to expression of osteogenic markers. This behavior can be recapitulated by culturing cells on patterns that induce cell contractility (as demonstrated herein); therefore this type of arrangement leads to enhancing osteogenic differentiation.

The contacting time for the tips can be from about 0.001 s to about 60 s, depending upon the amount of patterning composition desired in any specific point on a surface. The contacting force can be controlled by altering the z-piezo of the piezo scanner or by other means that allow for controlled application of force across the tip array.

The surface can be contacted with a tip array a plurality of times, wherein the tip array, the surface or both move to allow for different portions of the surface to be contacted. The time and pressure of each contacting step can be the same or different, depending upon the desired pattern. The shape of the indicia or patterns has no practical limitation, and can include dots, lines (e.g., straight or curved, formed from individual dots or continuously), a preselected pattern, or any combination thereof.

The indicia resulting from the disclosed methods have a high degree of sameness, and thus are uniform or substantially uniform in size, and preferably also in shape. The individual indicia feature size (e.g., a dot or line width) is highly uniform, for example within a tolerance of about 5%, or about 1%, or about 0.5%. The tolerance can be about 0.9%, about 0.8%, about 0.7%, about 0.6%, about 0.4%, about 0.3%, about 0.2%, or about 0.1%. Non-uniformity of feature size and/or shape can lead to roughness of indicia that can be undesirable for sub-micron type patterning.

The feature size can be about 10 nm to about 1 mm, about 10 nm to about 500 μm, about 10 nm to about 100μm, about 50 nm to about 100μm, about 50 nm to about 50 μm, about 50 nm to about 10μm, about 50 nm to about 5μm, or about 50 nm to about 1μm. Features sizes can be less than 1μm, less than about 900 nm, less than about 800 nm, less than about 700 nm, less than about 600 nm, less than about 500 nm, less than about 400 nm, less than about 300 nm, less than about 200 nm, less than about 100 nm, or less than about 90 nm.

### EXAMPLES

Large area polymer pen lithography was used to pattern substrates with nanoscale extracellular matrix protein features and to identify cues that can be used to direct cytoskeletal organization in human mesenchymal stem cells. This nanopatterning approach was used to identify how anisotropic focal adhesions around the periphery of symmetric patterns yield an organized and contractile actin cytoskeleton. The examples that follow show that anisotropic and periodic cues that increase cell contractility within a circular shape redirect cell differentiation from an adipogenic to an osteogenic fate. Together, these experiments demonstrate a programmable approach for using sub-cellular spatial cues to control cell behavior within defined geometries.

### Example 1

### Methods/Results

Substrate Preparation. Glass slides (1.9 cm×1.9 cm, 0.5 mm thick, Ted Pella) were sonicated for 30 min, rinsed in ethanol and dried under a stream of N₂. They were mounted in an electron-beam evaporator (Lesker) and when vacuum reached 2×10⁻⁷ mTorr, 5 nm of Ti and 35 nm of Au were evaporated.

Patterning of cell attachment sites. Polymer pen arrays were prepared using conventional photolithography techniques according to published methods.²³ The pen arrays were coated with an ethanolic solution of 10 mM MHA (16-mercaptohexadecanoic) (Sigma) for 2 min and dried under N₂. After mounting the Au substrate and pen array on the PPL system (Tera Fab M Series, Tera Print), the chamber humidity was held at 45% for patterning. For printing of MHA features on the surface, specific patterns were programmed (FIG. 1B for examples) into the software with tip-substrate dwell times of 2 s allowing for transfer of ink to the Au coated glass slide (FIG. 1A).

The patterned substrates were then immersed in an ethanolic solution of 10 mM 1-mercapto-11-undecyl hexa(ethylene glycol) (Sigma) for 1 hour to reduce nonspecific protein adsorption (FIG. 1A). After rinsing with ethanol and drying with N₂, the substrates were exposed to 50 μg/mL of human plasma fibronectin (Millipore) in phosphate buffered saline (PBS) and shaken overnight at 4° C. (FIG. 1A). The fibronectin serves to facilitate the formation of focal adhesions between cells and the substrate, which is used to define cell shape.

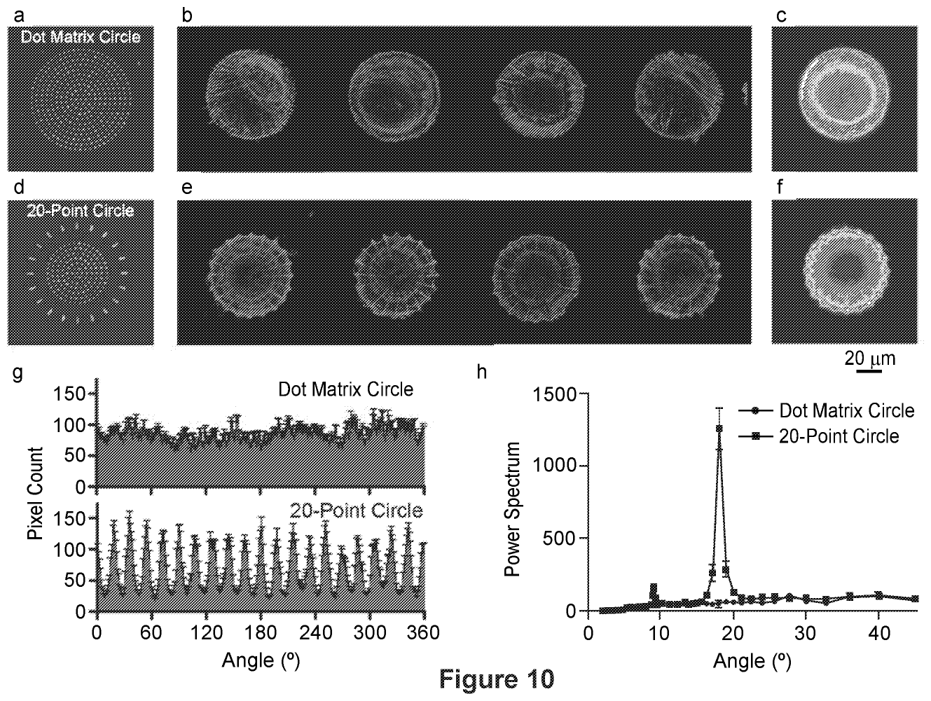

To confirm successful patterning, some substrates were sacrificed for imaging. Prepared and patterned glass slides treated with fibronectin were stained and visualized with fluorescence microscopy. The substrates were treated with an rabbit anti-fibronectin antibody (ThermoFisher) and shaken overnight at 4° C. in PBS. The substrates were rinsed with PBS and treated with an AlexaFluor568 goat anti-rabbit (ThermoFisher) secondary antibody for 1 hour at room temperature. The patterns were visualized on an Axio Imager Microscope (Zeiss) (FIG. 10).

Cell Culture on Substrates. Human MSCs (Lonza) were cultured at 37° C. with 5% CO₂ in MSC growth medium supplemented with MSC growth supplements (Lonza), L-glutamine (Lonza), and gentamycin sulfate amphotericin-1 (Lonza). The cells were used before passage 2. For chemical induction of differentiation, cells were cultured in osteogenic induction media (Lonza), which is composed of MSC growth medium supplement, L-glutamine, penicillin/streptomycin, and β-glycerophosphate (Lonza). Approximately 10,000 cells were seeded per substrate, corresponding to approximately 3,000 cells/cm².

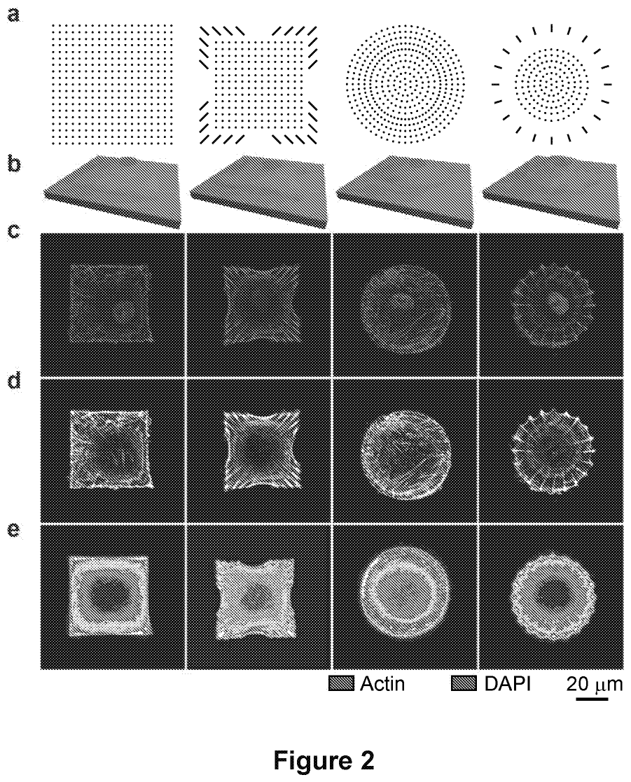

Immunofluorescence and Confocal Microscopy. For visualization of the actin cytoskeleton of cells on patterns, cells cultured on substrates were fixed in 3.7% paraformaldehyde in PBS for 12 minutes and then gently washed three times with PBS. Cells were permeabilized using 0.3% triton X-100 in PBS for 1 minute and blocked with a 0.1% Triton X-100 in PBS solution with 3% of bovine serum albumin for 1 hour. Next, samples were labeled for actin using Alexa Fluor 568-labeled phalloidin (ThermoFisher) according to manufacturer's instruction for actin labeling. Samples were gently washed three times in PBS and mounted onto glass coverslips using Prolong Gold Antifade reagent with DAPI (ThermoFisher). Cells were imaged using a Zeiss LSM 800 inverted laser-scanning confocal microscope with a 63X oil-immersion objective. Confocal stacks of the cells were acquired and maximum intensity projections were generated of each stack for analysis (FIG. 2C,D).

To determine if focal adhesions were directing cytoskeletal organization, focal adhesions were stained using an Anti-Vinculin antibody (Abcam) by shaking at 4° C. overnight.

Image Analysis. To generate heatmaps, images of fluorescence images of fixed/stained cells were aligned, stacked, averaged and pseudo-colored to represent regions of high and low density using ImageJ (FIG. 2E).

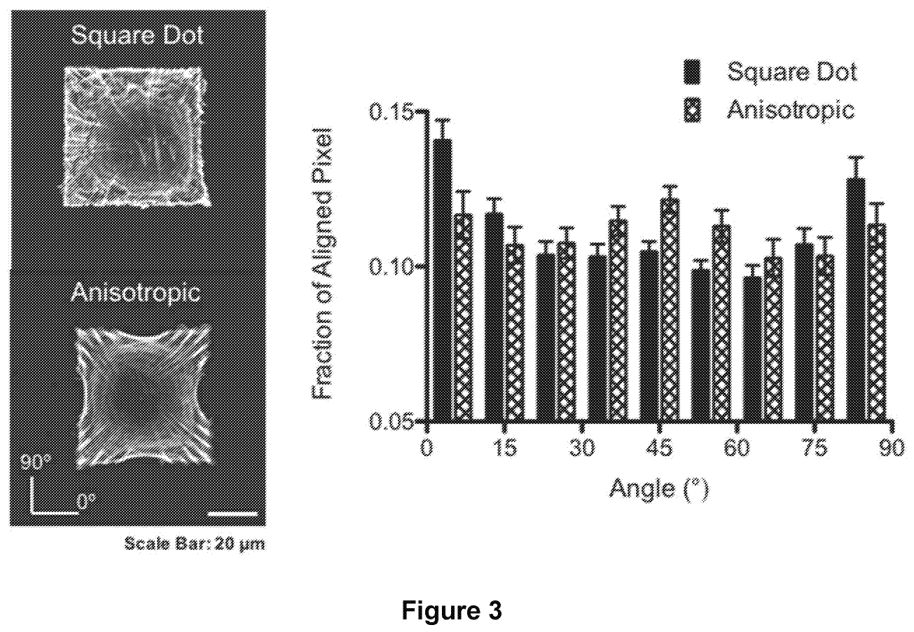

The orientation of actin fibers in cell micrographs was analyzed using a custom Matlab script that determines orientation using a modified version of a previously reported gradient analysis methods.²⁴ Briefly, a 5×5 Sobel Filter was used to detect changes in fluorescence gradients in the X and Y. The X and Y components of the gradient were then utilized to determine the orientation and magnitude of the gradient for each pixel. To reduce noise from weak or non-specific staining, a grayscale threshold, as determined using Otsu's method, was applied. For square and anisotropic square shaped patterns, fibers were detected within the entire cell as well as those within a region of interest (ROI) that was drawn to exclude fibers around the edge of the cell. Histograms of pixel orientation were generated for each cell after grouping fiber orientations along folding mirrored axes (e.g. −45° and)45° (FIG. 3). The anisotropic cell patterns (Column 2, FIG. 2), show greater alignment along the long axis (FIG. 3) compared to the square dot pattern (Column 1, FIG. 1).

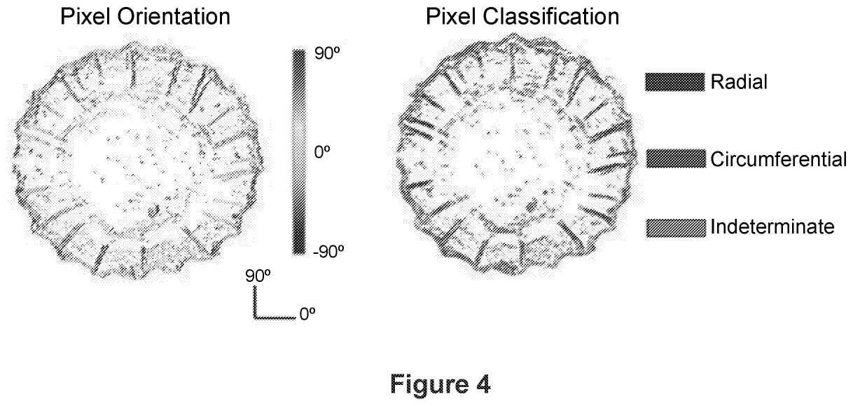

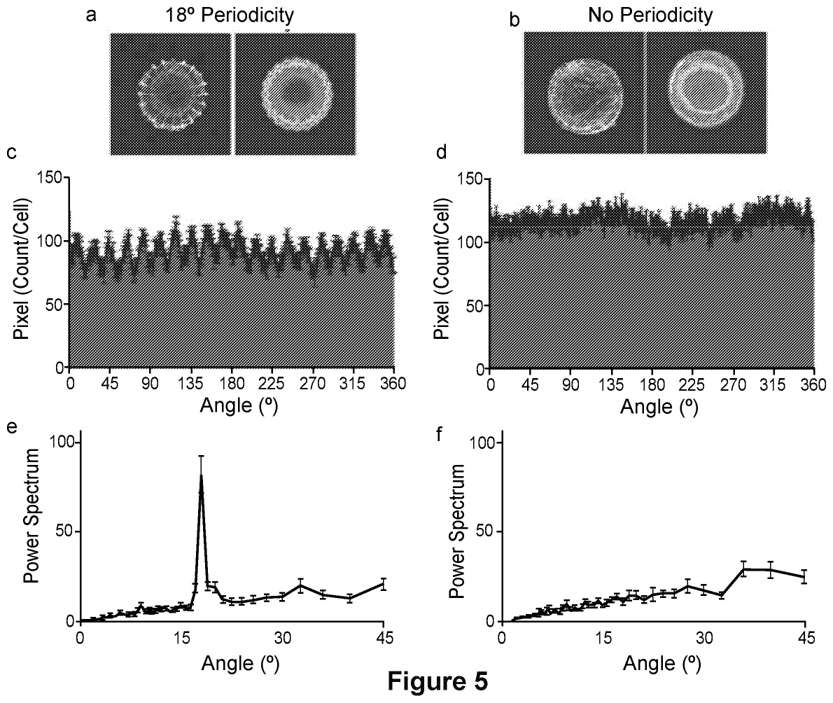

For detection of fibers on the circle (FIGS. 5a) and 20-point star (FIG. 5b) patterns, the center of the cell was detected by fitting a circle around the cells to identify the centroid. The pixel alignment was determined by comparing the detected pixel orientation to the radial coordinates of the pixel compared to the detected centroid. The fibers were classified as either radial (±30° from the radial coordinate), circumferential (90±30° from the radial coordinate), or indeterminate (FIG. 4b). Histograms of the distribution of the fibers were then generated (FIG. 5c,d). Fast Fourier transform (FFT) analysis was used to determine the angular distribution of radial fibers within a cell (FIG. 5E,F). The 20-point star shows prominent peaks at 18 degree periodicity as expected based on the pattern, while the circle pattern shows no prominent modes at lower angular frequencies. These results suggest that the use of 20-point star pattern results in greater uniformity of actin fibers in the radial direction.

Quantitative RT-PCR. Cells were harvested after one week of growth and mRNA expression was performed using TaqMan Gene Expression Cells-to-Ct Kit (Life Technologies) following the manufacturer's protocol. PCR was performed on the cDNA using Taqman primers for PPARγ, OCN, RUNX2, LPL, ALPL, GAPDH on a Roche Light Cycler 480 II System following manufacturer's instructions. The relative abundance of the mRNA levels for the genes investigated was normalized to glyceraldehyde 3-phosphate dehydrogenase (GAPDH) expression and compared to untreated cells to determine expression levels. Validated primers were obtained from Life Technologies.

### Example 2

### Methods

Substrate Preparation. Glass slides (1.9 cm·1.9 cm, 0.5 mm thick, Ted Pella) were sonicated for 30 minutes, rinsed in ethanol, and dried under a stream of N₂. They were mounted in an electron-beam evaporator (Lesker) and when vacuum reached 2×10⁻⁷ mTorr, 5 nm of Ti and 35 nm of Au were evaporated. Polymer pen arrays having a pen-to-pen distance of 150 μm were prepared using conventional photolithography techniques according to published methods [Eichelsdoerfer et al., Nat. Protoc. 8, 2548-2560 (2013)]. Arrays were coated with an ethanolic solution of 10 mM MHA (16-mercaptohexadecanoic) (Sigma) solution for 2 minutes and dried under N2. After mounting the Au substrate and pen array on the PPL system (Tera Fab M Series, Tera Print), the chamber humidity was held at 45% for patterning. Patterns were programmed in the software with tip-substrate dwell times of 2 seconds. Feature size and quality were confirmed by sacrificing a portion of the substrate, etching Au in the unpatterned areas with a mixed aqueous solution of 13.3 mM Fe(NO₃)₃ and 10 mM thiourea, and observing the results under an optical microscope. The patterned substrates were then immersed in an ethanolic solution of 10 mM 1-mercapto-11-undecyl hexa(ethylene glycol) (Sigma) solution for 1 hour to reduce non-specific protein adsorption. After rinsing with ethanol and drying with N₂, the substrates were exposed to 50 μg/mL of human plasma fibronectin (Millipore) in phosphate buffered saline (PBS) and shaken overnight at 4° C.

Cell Culture. Human MSCs (Lonza) were cultured at 37° C. with 5% CO₂ in Basal growth medium supplemented with MSC growth supplements (Invitrogen), L-glutamine (Invitrogen), and gentamycin sulfate amphotericin-1 (5 μg/ml; Invitrogen). The cells were used between passage 2 and 3. For chemical induction of differentiation, cells were cultured in mixed media (1:1 osteogenic:adipogenic induction media, PromoCell; 0.5 μg/mL, gentamycin). Approximately 20,000 cells were seeded per substrate. Mycoplasma contamination was monitored using MycoAlertPLUS (Lonza) following the manufacturer's instructions.

Immunofluorescence. hMSCs were cultured on patterned substrates overnight and then fixed in 3.7% paraformaldehyde in PBS for 10 minutes. After gently rinsing thrice with PBS, cells were permeabilized using 0.3% triton X-100 in PBS for 1 minute and blocked with a 0.1% Triton X-100 in PBS solution with 3% of bovine serum albumin for 1 hour. For immunofluorescence staining of focal adhesions and actin, primary antibody labeling for vinculin was performed in 1% bovine serum albumin (BSA) in PBS overnight at 4° C. with mouse-anti-vinculin (1:500, Abcam AB18058), followed by secondary antibody labeling using Alexa-Fluor 647 labeled goat anti-mouse (1:250, ThermoFisher A21236) for 1 hour at room temperature. Samples were rinsed at least twice with 1×PBS and then actin-labeled using Alexa Fluor 568-labeled phalloidin (ThermoFisher) for 30 minutes at room temperature. Samples were gently washed three times in PBS and mounted onto glass coverslips using Prolong Gold Antifade reagent with DAPI (ThermoFisher).

For immunofluorescence staining of myosin, samples were incubated in a solution containing an antibody produced in rabbit against non-muscle myosin Ila conjugated to Alexa Fluor 488 (1:750, Abcam AB204675) and 1% BSA in PBS for 1 h at 4° C. Vinculin was stained as described above.

Histology. hMSCs were seeded on patterned substrates and cultured for 6 days in the presence of mixed (adipogenic and osteogenic) media. Samples were then rinsed twice with PBS and fixed with 3.7% formaldehyde for 10 minutes. After rinsing twice with PBS, samples were permeabilized with a solution of 60% isopropanol in DI H₂O and stained with Oil Red O (Sigma, 60% isopropanol in DI H₂O) for 30 minutes at room temperature. Samples were then rinsed once with 60% isopropanol and then once with PBS and stained for alkaline phosphatase (StemTAG, Cell Biolab, Inc) for 1 hour at room temperature. Samples were rinsed twice with PBS and briefly rinsed in DI H₂O before mounting onto glass coverslips using Prolong Gold Antifade reagent containing DAPI. All substrates were imaged using a DAPI filter set and phase contrast microscopy using a 10X objective (Zeiss LSM 800).

Microscopy Image Analysis. To generate heatmaps, images of fluorescence images of fixed/stained cells were aligned, stacked, averaged and pseudo-colored to represent regions of high and low density using ImageJ. The orientation of actin fibers in cell micrographs was analyzed using a custom Matlab script that determines orientation using previous reported gradient analysis methods. Briefly, a pixel variance method was used to determine local fiber orientation and magnitude as previously described [Quinn et al., J. Biomed. Opt. 18, 046003 (2013)]. A grayscale threshold, as determined using Otsu's method [Otsu et al., IEEE Trans. Syst. Man. Cybern. 9, 62-66 (1979)], was applied to the gradient images. To eliminate imaging derived bias from confocal scanning, randomly selected images were rotated along their axis of symmetry. For square and anisotropic square shaped patterns, fibers were detected within the entire cell as well as those within a region of interest (ROI) that was drawn to exclude fibers around the edge of the cell. Histograms of pixel orientation were generated for each cell after grouping fiber orientations along mirrored axes (e.g., −45° and)45°.

For detection of fiber orientation in circular patterns (radial vs. circumferential), the center of the cell was detected by fitting a circle around the cells (details for this procedure are discussed herein below) to identify the centroid. The pixel alignment was determined by comparing the detected pixel orientation to the radial coordinates of that pixel relative to the cell centroid. The fibers were classified as either radial (±30° from the radial coordinate), circumferential (90±30° from the radial coordinate), or indeterminate (fiber not fitting within the first 2 groupings). Fast Fourier transform (FFT) analysis was used to determine the angular periodicity of the fibers within a cell.

Color deconvolution on phase contrast images was performed as previously described [Kilian et al., Proc. Natl. Acad. Sci. U.S.A. 107, 4872-4877 (2010)] using ImageJ (described in detail herein below). Briefly, the colors were deconvoluted to purple and red channels based upon visual inspection. An intensity cutoff was applied, and the images were binarized (purple and red) to determine cell fate. Cells containing purple were scored as osteocytes while those were red scored as adipocytes.

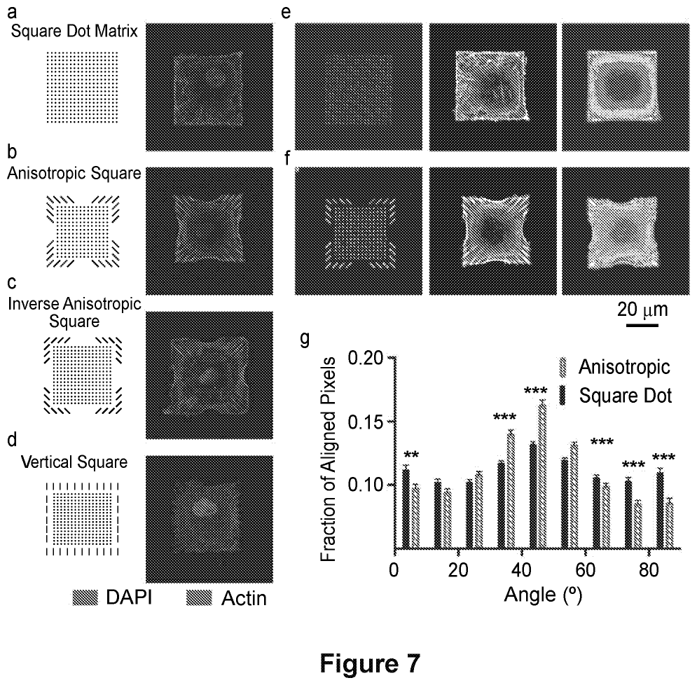

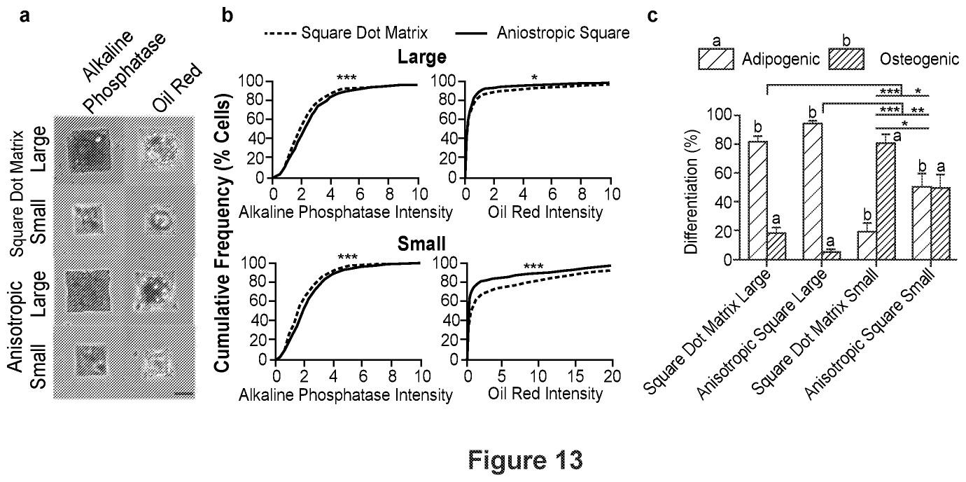

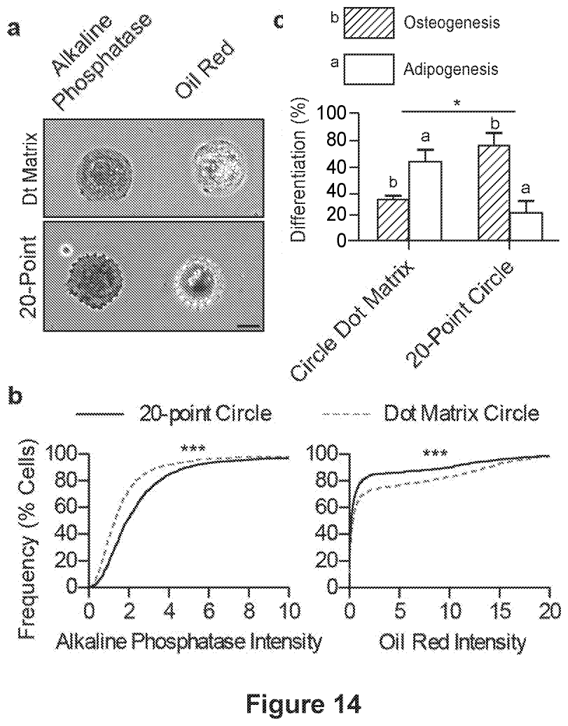

Statistical Analysis. All data were analyzed using Graph Pad PRISM 5 (Graph Pad Software, Inc.). For analysis of fiber orientation, the distribution of orientations was assessed using a two-way ANOVA with Bonferroni post hoc analysis. Cell differentiation was assessed using a one-way ANOVA with Tukey HSD post hoc analysis for the squares and a t-test for the circles. The distribution oil red and alkaline phosphatase staining was compared using Mann-Whitney U test. Statistical parameters are as follows: FIG. 7g (F_{interaction}=18.19, F=67.65); FIG. 13b (Large: Alkaline phosphatase, p=0.0005; Oil Red, p=0.0330; Small: Alkaline phosphatase, p<0.0001; Oil Red, p<0.0001); FIG. 13c (F=31.37); FIG. 14b (p=0.0246); FIG. 14c (p<0.0001 for both).

Centroid Fitting. The centroid of circular cells was found using a custom script in MATLAB R2017A. Exported images of actin cytoskeleton were imported into the program. Cell interiors were filled and then fit to disk using the Regionprops function. The centroid of the fitted disk was used to evaluate the relative fiber orientation.

Color Deconvolution. Cells on patterned surfaces were imaged with color phase contrast microscopy with a 10× objective (LSM 800, Carl Zeiss). Single cells were identified by counting the number of nuclei in fluorescence images using a DAPI filter set. 200×200 pixel or 150×150 pixel color images of each cell were then exported, color deconvoluted, and analyzed using the FIJI package of ImageJ (https://imagej.net/Fiji/Downloads). The background intensity of each image was corrected using a pseudo-flatfield correction with a blurring radius of 40 pixels (BioVoxxel Toolbox). Color vectors were assigned using the color deconvolution plugin within ImageJ. The images were then binarized using a set threshold based on visual examination and the intensity of the binarized images was measured for each channel.

### Results

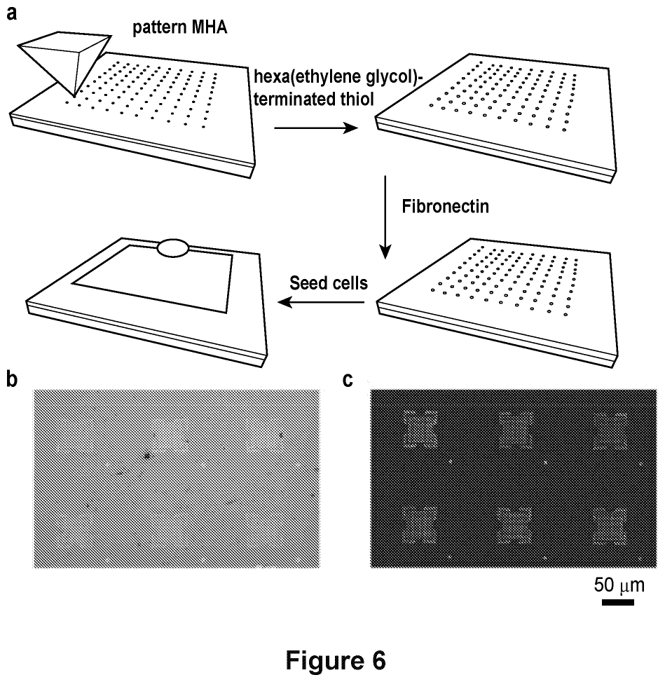

Generating Arbitrary Patterns for Cell Adhesion and Differentiation Studies Using PPL. Polymer pen lithography (PPL) was used to rapidly prepare substrates having nanoscale patterns of fibronectin with defined geometries. Pen arrays consisting of approximately 10,000 polymeric tips were inked with 16-mercaptohexadecanoic acid (MHA) and loaded into a PPL instrument. Patterns of MHA features were generated on gold-coated glass slides (FIG. 6a). To validate that patterns were printed successfully, a portion of the gold-coated glass substrates was chemically etched, and the resulting MHA printed substrates were visualized with optical microscopy (FIG. 6b). Next, the MHA patterned slides were treated with an ethanolic solution containing a hexa(ethylene glycol)-terminated alkanethiol, which backfilled the unmodified regions of gold with a chemically inert monolayer [Giam et al., Proc. Natl. Acad. Sci. U.S.A. 109, 4377-4382 (2012)] and which prevents the non-specific protein adsorption in these experiments. Fibronectin was then introduced on the surfaces to allow it to adsorb to the MHA features (FIG. 6a). Fluorescence micrographs confirm the discrete localization of antibody-labeled fibronectin on the surface in an arrangement defined by the MHA features (FIG. 6c). This process was used to prepare a range of substrates having several arrangements of fibronectin features for subsequent studies of cytoskeletal organization in adherent cells.

Cell Cytoskeleton Organization on Square Geometries. In a first example, an array of square features (54×54 μm²) was printed, each consisting of a dot matrix of 19×19 square fibronectin features of approximately 750 nm dimension (FIG. 7a,e). Human mesenchymal stem cells (hMSCs) were cultured on a substrate that presents a uniform distribution of isotropic fibronectin features within the square geometry and which serves as a benchmark for identifying the influence of anisotropic fibronectin features. After 16 hours of culture, the actin cytoskeleton was fixed and stained with a phalloidin-conjugated fluorophore and then the hMSCs were imaged using confocal microscopy. Heatmaps were generated by superimposing fluorescence micrographs of the actin cytoskeleton; these heatmaps confirmed that the square dot matrix pattern indeed does not induce any directional assembly of actin filaments (FIG. 7e, right panel). The distribution of stress fibers, however, localized along the periphery of the patterned square shape in most hMSCs.

Next, substrates were prepared that were patterned with asymmetric features, to ask whether the orientation and aspect ratio of anisotropic features could control the cytoskeleton, while maintaining constant cell size and shape. Specifically, the experiment queried whether the direction of actin stress filaments would correspond to the orientation of the anisotropic focal adhesions. Hence, a set of unique patterns was designed and printed having: 1) a center region presenting features arranged in a uniform square dot matrix that would provide sufficient ECM to support cell attachment and 2) a region along the periphery of the pattern where ligand arrangement and aspect ratio are varied (FIGS. 7b-d). Importantly, the overall shape of this pattern, and therefore of cells attached to the pattern, still maintained a 1:1 aspect ratio.

Fluorescence micrographs of actin fibers revealed that the anisotropic fibronectin features along the perimeter of the square shape could reinforce the alignment of actin fibers when they were aligned with the diagonal axes of the overall shape (FIG. 7b). For cells seeded on the inverse anisotropic square patterns where the fibronectin features are oriented perpendicular to the diagonal axis of the cell, actin fibers still preferentially aligned diagonally across the cell (FIG. 7c). In contrast, features arranged vertically around the periphery did not induce actin fibers to arrange along the edges of the pattern (FIG. 7d). Instead, these cues disrupted the organization of the cytoskeleton observed in the parent pattern.

These results suggested that the cues associated with a square cell shape dominate over those associated with the nanoscale fibronectin features; the corners of square shapes, for example, have been shown to be sites for recruitment of focal adhesions and associated stress filaments. Cells on all three patterns having different orientations of the anisotropic fibronectin features were found to share a similar organization of the cytoskeleton that is determined by the global cell shape and not the orientation of the anisotropic fibronectin features. Although, the pattern having anisotropic fibronectin features was found to have aligned with the diagonal axis shows the most organized cytoskeleton (FIG. 7b).

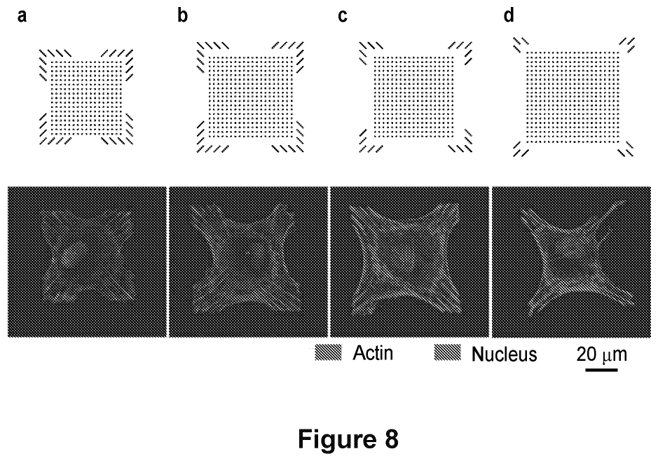

To quantitatively assess the role of anisotropic fibronectin feature orientation on actin organization, an in-depth analysis on these cells was performed (FIG. 7f). For this analysis, the distribution of actin fibers in hMSCs on the square dot matrix pattern was compared to those on the anisotropic square of the same size (FIG. 7e,f). Heatmaps of the average actin fluorescence intensity showed preferential alignment of fibers toward the interior of the cell on the square anisotropic pattern (FIG. 7f, right panel). When fiber orientation was quantified using a gradient detection method [Quinn et al., J. Biomed. Opt. 18, 046003 (2013)], the fiber alignment indeed increased along the long axes (+45° and −45°) for cells on anisotropic patterns compared to the square dot matrix (FIG. 7g). These results showed that the anisotropic arrangement of adhesion ligands supports the assembly of user-directed actin architectures. In addition, when the area of this pattern was increased—where the center region of the dot matrix pattern was increased from 30×30 to 40×40 μm², it was found that the anisotropic features had the same influence on cytoskeletal alignment (FIG. 8).

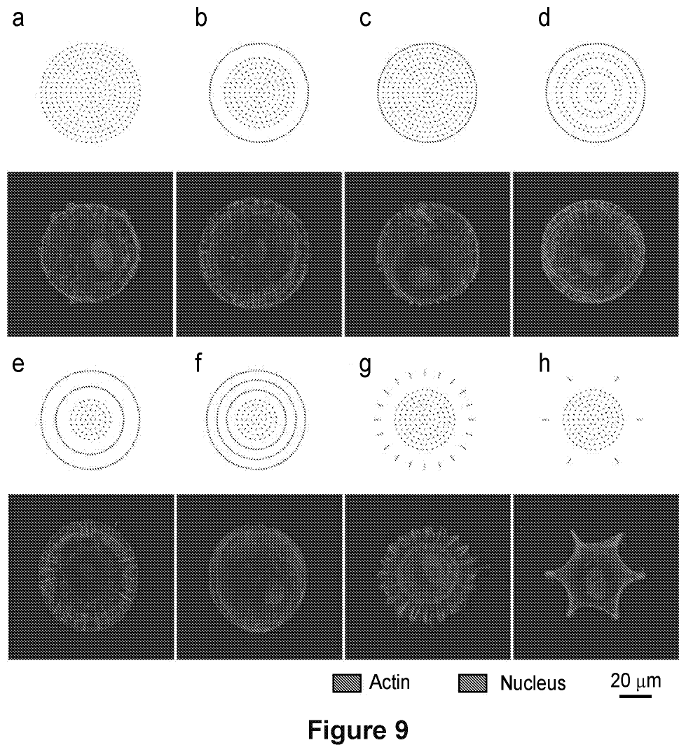

MSC Cytoskeleton Organization on Circular Geometries. As described above, the strong global cues of the square patterns—which lead to stress filaments aligned along the diagonals of the cell—dominated over the anisotropic fibronectin features. Hence, these experiments were repeated using circular patterns, which have been demonstrated to only weakly direct the assembly of the cytoskeleton [Kilian et al., Proc. Natl. Acad. Sci. U.S.A. 107, 4872-4877 (2010)]. In this way, it could be determined whether the anisotropic features, absent global shape cues, could direct cytoskeletal assembly. Patterns having a radius of 31 μm were prepared because they would have the same area as the square patterns described above. Again, a set of patterns was generated to examine parameters that may affect actin fiber alignment (FIG. 9a-h). Previous studies have shown that circumferential and chordal fibers are the primary actin orientations within cells seeded on microcontact printed circular geometries [Tee et al., Nat. Cell Biol. 17, 445-457 (2015)]. Therefore, the distribution of fibronectin was varied in two ways: 1) concentric rings were introduced within the circle and 2) periodic, anisotropic features consisting of anisotropic dot lines were placed around the periphery. In addition, all patterns contained a central region presenting fibronectin features arranged in a circular dot matrix that supported cell attachment and spreading.

Fluorescence micrographs confirmed the formation of circumferential and radial actin fibers within hMSCs on all the circular shapes (FIG. 9a-h). However, circular shapes with concentric rings of fibronectin features did not significantly change the circumferential fiber distribution within the cells (FIG. 9b,d-f). On the other hand, when anisotropic features were introduced radially around the circle, actin fibers assembled in a periodic fashion reflecting the pattern design (FIG. 9g). It was also noted that a sparse spacing between peripheral features promoted different cell geometries that no longer reflect a circular shape (FIG. 9h). From this library, a periodic pattern consisting of 20 anisotropic features arranged around the cell periphery that promoted the formation of distinct radial fibers was identified (FIG. 10d).

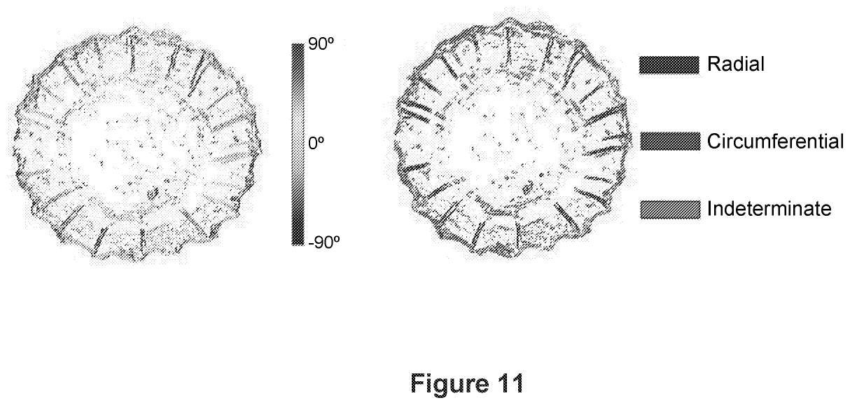

Cells seeded on the 20-point circle were compared to those seeded on patterns presenting symmetric features arranged in a circular shape (FIG. 10a,b,d,e). Heatmaps of the average actin intensity reveal a periodicity in the radial fiber distribution within cells on 20-point circles that is not observed when cells are seeded on dot matrix circles (FIG. 10c, f). In order to assess the periodicity of the fibers, the local fiber orientation was determined, and the fibers were classified as either radial or circumferential depending on the local orientation of the fiber relative to the centroid of the cell (FIG. 11). The angular distribution of the radial fibers had a clear periodicity within cells on the 20-point circular pattern (FIG. 10g). Fast Fourier transform (FFT) analysis of the fiber distribution confirms an 18° periodicity for the 20-point circles (FIG. 10h). In contrast, no periodicity was measured within cells on a dot matrix pattern (FIG. 10h).

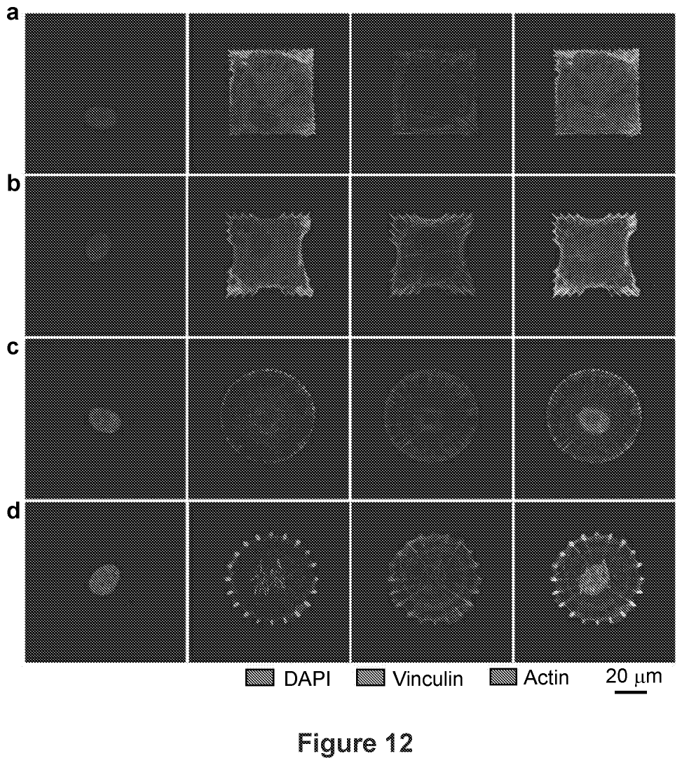

Focal Adhesion Distribution on Patterned Surfaces in Response to Anisotropy. Since focal adhesion complexes link the actin cytoskeleton to the ECM, their distribution within cells seeded on anisotropic, periodic, and dot matrix patterns was examined. Immunofluorescence micrographs of vinculin, a focal adhesion protein, showed that the focal adhesions primarily form along the periphery of the cell in locations defined by the underlying pattern (FIG. 12a-d). Significantly, the focal adhesion geometry reflected the shape of the underlying pattern, and more importantly, the orientation of the actin stress filament was aligned with the fibronectin feature. These results highlighted that the geometry and distribution of adhesive cues lead to focal adhesion formation at desired locations with programmed anisotropy, which directs the actin cytoskeletal architecture.

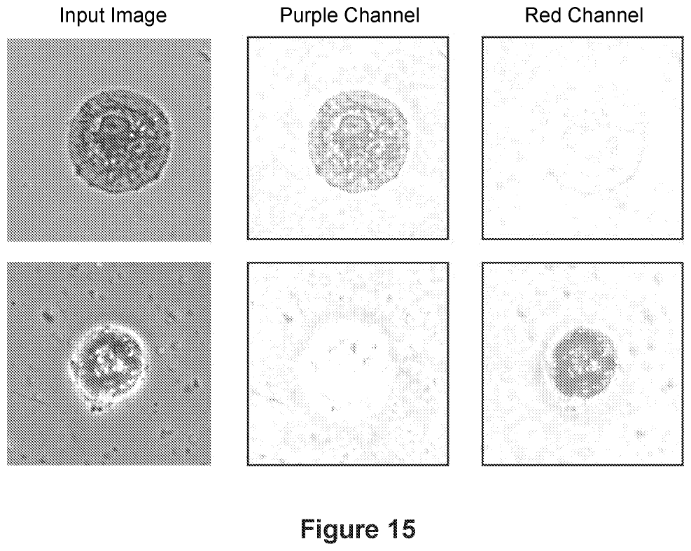

Modulating hMSC Fate in Response to Focal Adhesion-Defined Actin Arrangements. With the demonstration that anisotropic fibronectin features direct focal adhesion formation and actin orientation, it was next investigated whether focal adhesion anisotropy could be used to modulate differentiation independent of cell shape. To evaluate lineage-specific differentiation, hMSCs were cultured on patterns having either anisotropic (anisotropic squares and 20-point circles) or isotropic (square dot matrix and circle dot matrix) ligand arrangements in mixed osteogenic and adipogenic induction media. After 6 days, cells were fixed and stained for osteogenic and adipogenic markers. Cells that stained purple due to alkaline phosphatase activity were counted as osteoblasts while those that stained red from oil droplets (Oil Red O) were counted as adipocytes (FIGS. 13a, 14a). Cells were also stained with the nuclear dye DAPI and cells that occupied a pattern with a second cell were not scored. Color deconvolution was performed on images of single cells to separate and binarize the purple and red channels (FIG. 15). The intensity of the binarized images was used to score cells as staining either positive or negative for each marker (FIGS. 13b, 14b).

Cells grown on both square patterns—the square dot matrix and anisotropic—showed a strong preference (>80%) for osteogenesis when cultured on larger patterns (54×54 μm²; FIG. 13b-c), as was expected for the strong cue that corners present to a cell and the pro-osteogenic effect of greater spread area [Kilian et al., Proc. Natl. Acad. Sci. U.S.A. 107, 4872-4877 (2010); McBeath et al., Dev Cell 6, 483-495 (2004)]. To determine whether focal adhesion anisotropy could synergistically enhance differentiation, the overall pattern area was decreased to reduce the osteogenic cue (36×36 μm²; FIG. 13b-c), and a stronger preference (50%) for osteogenesis for cells seeded on the anisotropic square patterns compared to those grown on the square dot matrix patterns (20%, FIG. 13b-c) was found. These results illustrated how anisotropic arrangements of ligands can synergize with global cell shape to promote an osteogenic fate.

Next, this experiment was repeated for cells cultured on circular patterns. These shapes lack any corners along their perimeter and are known to limit the organization of a contractile cytoskeleton, leading to adipogenic fates [McBeath et al., Dev Cell 6, 483-495 (2004)]. As expected, a preference for adipogenesis for cells grown on dot matrix circles (70%, FIG. 14b-c) was observed. In contrast, cells grown on the 20-point circle patterns preferentially differentiated into osteogenic fates (80%, FIG. 14b-c). This significant result demonstrated that the anisotropic fibronectin features, while maintaining a constant shape and size, can redirect cells from primarily adipogenic to osteogenic fates.

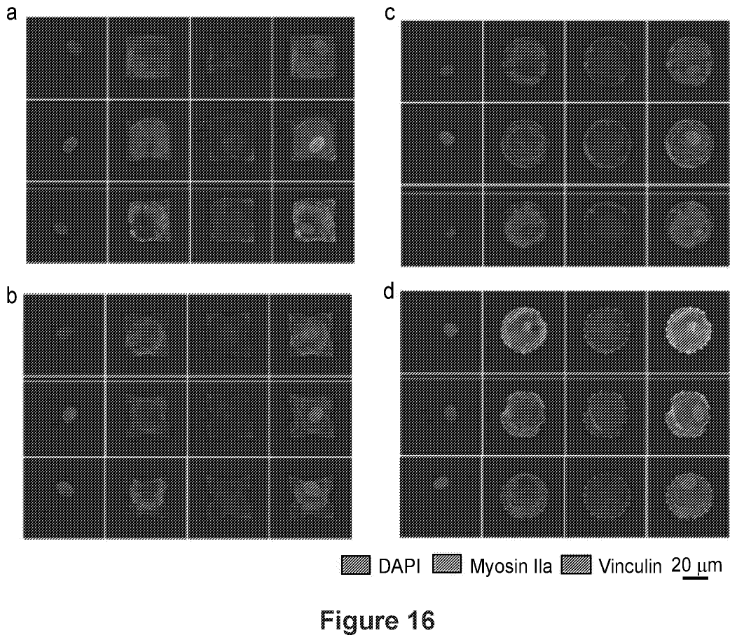

Previous work suggested that the enhanced osteogenesis results from an increased contractility of the acto-myosin cytoskeleton in the cell [Kilian et al., Proc. Natl. Acad. Sci. U. S. A. 107, 4872-4877 (2010); McBeath et al., Dev Cell 6, 483-495 (2004); Ward et al., Stem Cells Dev. 16, 467-479 (2007); Graziano et al., PLoS One 22007)]. Therefore, how the anisotropic arrangement of ligands affected the formation of contractile actomyosin fibers (FIG. 16) was examined. In order to evaluate the actomyosin contractility of hMSCs, these cells were cultured on the square, anisotropic square, circular, and 20-point circle patterns. After 16 hours of culture, cells were fixed and then stained for vinculin, focal adhesions, and myosin IIa. Fluorescent micrographs revealed that myosin Ila was localized along the fibers that have been directed by the anisotropic focal adhesions, suggesting that these user-defined fibers are indeed contractile (for both the square anisotropic and 20-point circle patterns). This observation is especially important for the circular geometry where the 20-point circle drastically altered the contractility by inducing the formation of contractile radial fibers not present in the dot matrix circle. Therefore, the propensity for these cells to differentiate to osteogenic lineages stems from enhanced contractility induced by the anisotropic ligand arrangement.

### Conclusions

This work investigated how anisotropic sub-micron patterns of fibronectin can be used to direct organization of the cytoskeleton. It is shown herein that arranging focal adhesions in peripheral areas of cells using nanopatterning enables the controlled reorientation of actin fibers within the interior of cells. The work is also significant because it provides a strategy for modulating actin fiber orientation independently of cell shape and allows one to confine cells on substrates and program the cytoskeleton in a predefined fashion. Indeed, the demonstration that sMSCs could be patterned in circular shapes but still undergo osteogenesis reveals the importance of these anisotropic features in directing cell function. The use of PPL to define discrete nanofeatures uniquely gives the ability to tailor focal adhesion formation over large areas in any arbitrary shape to modulate and study cell behavior. Although a single chemical cue was used in this study, multiplexing with PPL is also contemplated, opening up the opportunity to study the importance of multiple cues in combination with controlled morphology and contractility in influencing cellular fate. This new approach to cellular engineering enables the modulation of cell behavior in complex biological environments, such as those containing multiple different cell types. It also allows one to direct stem cells down different lineages within the same confined space to mimic tissue organization. This ability to control and direct cytoskeletal formation can be extended beyond stem cell differentiation to study and control other biological systems such as neurite formation and cancer metastasis.

### REFERENCES

1. Arnold, M.; Hirschfeld-Warneken, V. C.; Lohmuller, T.; Heil, P.; Blummel, J.; Cavalcanti-Adam, E. A.; Lopez-Garcia, M.; Walther, P.; Kessler, H.; Geiger, B.; Spatz, J. P., Induction of cell polarization and migration by a gradient of nanoscale variations in adhesive ligand spacing. Nano Lett 2008, 8 (7), 2063-9.

2. Letort, G.; Ennomani, H.; Gressin, L.; Thery, M.; Blanchoin, L., Dynamic reorganization of the actin cytoskeleton. F1000Res 2015, 4.

3. Delon, I.; Brown, N. H., Integrins and the actin cytoskeleton. Curr Opin Cell Biol 2007, 19 (1), 43-50.

4. Geiger, B.; Spatz, J. P.; Bershadsky, A. D., Environmental sensing through focal adhesions. Nat Rev Mol Cell Biol 2009, 10 (1), 21-33.

5. Kilian, K. A.; Bugarija, B.; Lahn, B. T.; Mrksich, M., Geometric cues for directing the differentiation of mesenchymal stem cells. Proc Natl Acad Sci USA 2010, 107 (11), 4872-7.

6. Shabbir, S. H.; Cleland, M. M.; Goldman, R. D.; Mrksich, M., Geometric control of vimentin intermediate filaments. Biomaterials 2014, 35(5), 1359-66.

7. Boujemaa-Paterski, R.; Galland, R.; Suarez, C.; Guerin, C.; Thery, M.; Blanchoin, L., Directed actin assembly and motility. Methods Enzymol 2014, 540, 283-300.

8. Selhuber-Unkel, C.; Erdmann, T.; Lopez-Garcia, M.; Kessler, H.; Schwarz, U. S.; Spatz, J. P., Cell adhesion strength is controlled by intermolecular spacing of adhesion receptors. Biophys J2010, 98 (4), 543-51.

9. Spatz, J. P.; Geiger, B., Molecular engineering of cellular environments: cell adhesion to nano-digital surfaces. Methods Cell Biol 2007, 83, 89-111.

10. Huang, J.; Grater, S. V.; Corbellini, F.; Rinck, S.; Bock, E.; Kemkemer, R.; Kessler, H.; Ding, J.; Spatz, J. P., Impact of order and disorder in RGD nanopatterns on cell adhesion. Nano Lett 2009, 9 (3), 1111-6.

11. Lohmuller, T.; Aydin, D.; Schwieder, M.; Morhard, C.; Louban, I.; Pacholski, C.; Spatz, J. P., Nanopatterning by block copolymer micelle nanolithography and bioinspired applications. Biointerphases 2011, 6 (1), MR1-12.

12. Aydin, D.; Schwieder, M.; Louban, I.; Knoppe, S.; Ulmer, J.; Haas, T. L.; Walczak, H.; Spatz, J. P., Micro-nanostructured protein arrays: a tool for geometrically controlled ligand presentation. Small 2009, 5(9), 1014-8.

13. Oakes, P. W.; Banerjee, S.; Marchetti, M. C.; Gardel, M. L., Geometry regulates traction stresses in adherent cells. Biophys J2014, 107(4), 825-33.

14. Ennomani, H.; Letort, G.; Guerin, C.; Martiel, J. L.; Cao, W.; Nedelec, F.; De La Cruz, E. M.; Thery, M.; Blanchoin, L., Architecture and Connectivity Govern Actin Network Contractility. Curr Biol 2016, 26 (5), 616-26.

15. Murrell, M.; Oakes, P. W.; Lenz, M.; Gardel, M. L., Forcing cells into shape: the mechanics of actomyosin contractility. Nat Rev Mol Cell Biol 2015, 16 (8), 486-98.

16. Thery, M.; Racine, V.; Pepin, A.; Piel, M.; Chen, Y.; Sibarita, J. B.; Bornens, M., The extracellular matrix guides the orientation of the cell division axis. Nat Cell Biol 2005, 7 (10), 947-53.

17. Thery, M.; Racine, V.; Piel, M.; Pepin, A.; Dimitrov, A.; Chen, Y.; Sibarita, J. B.; Bornens, M., Anisotropy of cell adhesive microenvironment governs cell internal organization and orientation of polarity. Proc Natl Acad Sci USA 2006, 103 (52), 19771-6.

18. Arnold, M.; Schwieder, M.; Blummel, J.; Cavalcanti-Adam, E. A.; Lopez-Garcia, M.; Kessler, H.; Geiger, B.; Spatz, J. P., Cell interactions with hierarchically structured nano-patterned adhesive surfaces. Soft Matter 2009, 5 (1), 72-77.

19. Giam, L. R.; Massich, M. D.; Hao, L.; Shin Wong, L.; Mader, C. C.; Mirkin, C. A., Scanning probe-enabled nanocombinatorics define the relationship between fibronectin feature size and stem cell fate. Proc Natl Acad Sci USA 2012, 109 (12), 4377-82.

20. Eichelsdoerfer, D. J.; Liao, X.; Cabezas, M. D.; Morris, W.; Radha, B.; Brown, K. A.; Giam, L. R.; Braunschweig, A. B.; Mirkin, C. A., Large-area molecular patterning with polymer pen lithography. Nat Protoc 2013, 8 (12), 2548-60.

21. Cabezas, M. D.; Eichelsdoerfer, D. J.; Brown, K. A.; Mrksich, M.; Mirkin, C. A., Combinatorial screening of mesenchymal stem cell adhesion and differentiation using polymer pen lithography. Methods Cell Biol 2014, 119, 261-76.

22. Cabezas, M. D.; Mirkin, C. A.; Mrksich, M., Nanopatterned Extracellular Matrices Enable Cell-Based Assays with a Mass Spectrometric Readout. Nano Lett 2017, 17 (3), 1373-1377.

23. Eichelsdoerfer, D. J.; Liao, X.; Cabezas, M. D.; Morris, W.; Radha, B.; Brown, K. A.; Giam, L. R.; Braunschweig, A. B.; Mirkin, C. A., Large-area molecular patterning with polymer pen lithography. Nature protocols 2013, 8 (12), 2548.

24. Baştürk, A.; Gunay, E., Efficient edge detection in digital images using a cellular neural network optimized by differential evolution algorithm. Expert Systems with Applications 2009, 36 (2), 2645-2650.

What is claimed is:

1. A method of making an oriented array, comprising:
printing a surface with an array of a cell adhesion ligand by (i) coating a polymer pen lithography (PPL) tip array with a monolayer reagent, (ii) printing the monolayer reagent at selected positions on the surface to form an array having a selected orientation of printed monolayer reagent, (iii) contacting the array of printed monolayer reagent with the cell adhesion ligand under conditions to immobilize the cell adhesion ligand to the surface at the printed monolayer reagent positions from step (ii), thereby forming the oriented array of the cell adhesion ligand.

2. The method of claim 1, wherein the monolayer reagent is mercaptohexadecanoic acid (MHA).

3. The method of claim 1 or claim 2, wherein the cell adhesion ligand is a protein, a peptide, or an antibody.

4. The method of claim 3, wherein the protein is an extracellular matrix (ECM) protein.

5. The method of claim 4, wherein the ECM protein is fibronectin, collagen, elastin, vitronectin, bone sialoprotein, or laminin.

6. The method of claim 3, wherein the peptide is an RGD, GFOGER (SEQ ID NO: 1), and/or YGISR (SEQ ID NO: 2) peptide.

7. The method of any one of claims 1-6, further comprising (iv) introducing a second monolayer that is a bio-inert region that inhibits association with the cell.

8. The method of any one of claims 1-7, wherein the surface comprises a multi-well plate.

9. The method of any one of claims 1-8, wherein the surface comprises gold, silver, silica, glass, quartz, a metal-oxide, or copper.

10. The method of any one of claim 1-9, wherein more than one cell is contacted with the surface.

11. The method of claim 10, wherein 2, 5, 10, 20, 50, or 100 cells are contacted with the surface.

12. The method of any one of claims 1-11, wherein the cell adhesion ligand is bound to the surface via a linker.

13. The method of claim 12, wherein the linker has a structure of formula I: 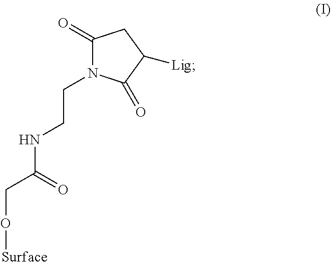
and Lig comprises the cell adhesion ligand.

14. The method of claim 12, wherein the surface comprises a monolayer.

15. The method of claim 14, wherein the monolayer comprises (i) the linker and (ii) an ethylene glycol and a C₂₋₂₀alkylene moiety.

16. The method of claim 14 or claim 15, wherein the monolayer is attached to the surface via a thiol bond.

17. The method of any one of claims 1-16, wherein the PPL tip array comprises a compressible elastomeric polymer comprising a plurality of non-cantilevered tips each having a radius of curvature of less than 1 μm and a common substrate comprising a compressible elastomeric polymer, the tip array and the common substrate mounted onto a rigid support and the tip array, common substrate, and rigid support together being at least translucent.

18. The method of claim 17, wherein the compressible elastomeric polymer comprises polydimethylsiloxane (PDMS).

19. A patterned array produced by the method of any one of claims 1-18.

20. A method of modulating cytoskeletal formation in a cell, comprising:
providing a nanoscale pattern of a cell adhesion ligand on a surface by (i) coating a polymer pen lithography (PPL) tip array with a monolayer reagent, (ii) printing the monolayer reagent at selected positions on the surface to form an array of a selected orientation of printed monolayer reagent, (iii) contacting the array of printed monolayer reagent with the cell adhesion ligand under conditions to immobilize the cell adhesion ligand to the surface at the printed monolayer reagent positions from step (ii), thereby forming an oriented array of the cell adhesion ligand;
contacting the surface with the cell and then culturing the cell to allow for cell growth;
wherein orientation of the array of the cell adhesion ligand modulates cytoskeletal formation in the cell.

21. The method of claim 20, further comprising (iv) introducing a second monolayer that is a bio-inert region that inhibits association with the cell.

22. The method of claim 20 or claim 21, wherein orientation of the array promotes uniform cell size and/or shape.

23. The method of any one of claims 20-22, wherein orientation of the array promotes differentiation of the cell.

24. The method of claim 23, further comprising contacting the cell with a growth and/or differentiation factor.

25. The method of claim 24, wherein the growth factor is h-insulin, TGF-β, VEGF, IL-3, IL-6, IL-11, EGF, FGF, Oct-3, Sox2, BMP, IGF, Activin, Wnt, or a combination thereof.

26. The method of claim 24 or claim 25, wherein the differentiation factor is dexamethasone, ascorbate, L-glutamine, B-glycerophosphate, indomethacin, 3-isobutyl-l-methyl-xanthine, or a combination thereof.

27. The method of any one of claims 20-26, wherein the cell is a stem cell, cancer cells, a neuronally-derived cell, or a combination thereof.

28. The method of claim 27, wherein the stem cell is a human mesenchymal stem cell (hMSC), a fibroblast, an induced-pluripotent stem cell (IPSO), an epidermal stem cell, a hemopoeitic stem cell, an embryonic stem cell, a neural stem cell, or a dermal stem cell.


SEQUENCE LISTING (3 sequences)

SEQ ID NO: 1 - protein, 6 aa, Artificial Sequence
  Synthetic Pepetide
  MISC_FEATURE (3)..(3): Xaa is Hydroxyproline
GFXGER

SEQ ID NO: 2 - protein, 5 aa, Artificial Sequence
  Synthetic Pepetide
YGISR

SEQ ID NO: 3 - protein, 4 aa, Artificial Sequence
  Synthetic Pepetide
GRTY